(19) United States
(12) Patent Application Publication
(10) Pub. No.: US 2026/0216139 A1
(43) Pub. Date: Jul. 30, 2026

(54) METHOD FOR TREATING NEUROLOGICAL DISEASES THROUGH GLIAL PRUNING REGULATION

(71) Applicant: Fred Hutchinson Cancer Center, Seattle, WA (US)

(72) Inventors: Aakanksha Singhvi, Seattle, WA (US); Stephan Raiders, Seattle, WA (US)

(73) Assignee: Fred Hutchinson Cancer Center, Seattle, WA (US)

(21) Appl. No.: 19/139,298

(22) PCT Filed: Dec. 14, 2023
(86) PCT No.: PCT/US2023/084125

§ 371 Date: Jun. 13, 2025

Related U.S. Application Data
(60) Provisional application No. 63/387,330, filed on Dec. 14, 2022.

(51) Int. Cl. A61K 31/416; A61K 31/12; A61K 31/5377

(52) U.S. Cl. CPC A61K 31/416; A61K 31/12; A61K 31/5377

(57) ABSTRACT

Methods for regulating aberrant glia-directed engulfment and active pruning of Neuronal Receptive Endings (NRE) are provided. Such methods comprise contacting a neuronal and/or glia cell with an effective amount of a composition which is effective in modulating at least one gene and/or protein associated with glia-directed engulfment and active pruning of Neuronal Receptive Endings. Such methods are useful in treating neurological disorders such as Parkinson's disease.

### CROSS-REFERENCE TO RELATED APPLICATION

This application claims the benefit of U.S. Provisional Patent Application No. U.S. 63/387,330, filed Dec. 14, 2022, the disclosure of which is hereby incorporated by reference in its entirety.

### STATEMENT OF GOVERNMENT LICENSE RIGHTS

This invention was made with Government support under NS118958, AG066574, and NS114222 awarded by National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P67WO_Seq_List_20231214.xml. The XML file is 4,675,801 bytes; was created on Dec. 14, 2023; and is being submitted electronically via Patent Center with the filing of the specification.

### BACKGROUND

The formation of mature neural circuits requires selective pruning of inappropriate synapses and strengthening of appropriate synaptic connections. This process is critical to normal brain maturation; disruptions are thought to contribute to neurological dysfunction. Glia, non-neuronal brain cells, contribute to this process. Aberrant uptake of neuron fragments by glia is associated with neuro-developmental and neuro-degenerative diseases such as epilepsy, Alzheimer's dementia, and cognitive decline.

Parkinson's disease (PD) is currently the second most prevalent neurodegenerative disease and is one of the world's fastest growing neurological disorders. One study estimates that by 2037, in the US alone, Parkinson's disease will affect more than 1.6 million people and have an economic burden exceeding $79 billion. Currently, no treatment exists to slow or terminate the disease progression of Parkinson's disease. Therefore, there remains a need for understanding the mechanisms involved in PD and for the development of effective therapeutics and other treatment options for treating, preventing, and/or delaying onset of PD.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some aspects, provided herein is a method for regulating an aberrant glia-directed engulfment and active pruning of Neuronal Receptive Endings (NRE), the method comprising contacting a neuronal and/or a glia cell with an effective amount of a composition effective in modulating at least one gene and/or protein associated with glia-directed engulfment and active pruning of Neuronal Receptive Endings (NRE).

In other aspects, provided herein is a method for treating a neurodegenerative disease in a subject in need thereof, the method comprising administering to the subject an effective amount of a therapeutic composition effective in modulating at least one gene and/or protein associated with glia-directed active pruning of Neuronal Receptive Endings (NRE).

In yet other aspects, provided herein is a method for treating, preventing, or delaying onset of Parkinson's Disease in a subject in need thereof, the method comprising administering to the subject a therapeutically effective amount of a composition effective in modulating at least one gene and/or protein associated with Parkinson's Disease (PD), wherein the gene and/or protein is expressed in a glia cell.

In additional aspects, provided herein is a method for the treatment of Parkinson's disease comprising administering to a subject in need thereof a composition effective in modulating the activity of Janus N-terminal Kinase (JNK) or an isoform thereof, wherein the composition is targeted for delivery to glia cells, and wherein the subject harbors a loss of function gene mutation in PARKIN or PINK1.

In further aspects, provided herein is a method for the treatment of Parkinson's disease comprising administering to the subject in need thereof a composition effective in modulating CED-10/Rac-1 or an isoform thereof, wherein the composition is targeted for delivery to the glia, and wherein the subject harbors a loss of function gene mutation in PARKIN or PINK1.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows an increase in engulfed fragments of pdr-1 and pink-1, thereby demonstrating PDR-1 and PINK-1 regulate engulfment;

FIG. 1B shows the size of “eaten” puncta in glia are unchanged, and thus the rate of eating is altered;

FIG. 2A shows LRK-1 regulates engulfment, as shown by the reduced number of fragments from Irk-relative to wildtype;

FIG. 2B shows DJR-1.2 regulates engulfment, as shown by the increased number of fragments of djr-1.2 relative to wildtype;

FIG. 3 shows JNK signaling regulates engulfment, wherein kgb-1 and kgb-2 have opposite effects on glial engulfment while jnk-1 has no effect, as demonstrated by a similar number of fragments identified for jnk-1 while kgb-1 and kgb-2 exhibit decreased and increased number of fragments, respectively;

FIG. 4A shows PINK-1 acts in glia, wherein expressing PINK-1 in glia rescues glial engulfment defects back to wild type levels, thereby demonstrating its function is required in glia;

FIG. 4B shows PINK-1 does not act in neurons, wherein expressing PINK-1 in neurons does not rescue glial engulfment defects back to wild type levels;

FIG. 5A shows PDR-1 acts in glia, wherein expressing PDR-1 in glia rescues glial engulfment, as demonstrated by a similar number of fragments identified upon glia rescue compared with wildtype and a decreased number of fragments as compared with pdr-1; over-expression of PDR-1 in glia also alters pruning levels compared to wild type;

FIG. 5B shows PDR-1 acts in glia, wherein glial PDR-1 rescues defects pdr-1; pink-1 double mutants, as demonstrated by rescue of double mutant defects upon over-expression of PDR-1 in glia;

FIG. 5C shows PDR-1 expression in neurons does not rescue the mutant defects;

FIG. 6A shows PDR-1 (Parkin) and PINK-1 are not in the same pathway, wherein PDR-1 and PINK-1 work independently to regulate glial engulfment, and losing both causes strong defects in glial engulfment, as demonstrated by the increased number of fragments for pdr-1; pink-1 relative to wildtype, for animals just prior to sexual maturation (pre-adult);

FIG. 6B shows PDR-1 (Parkin) and PINK-1 are not in the same pathway, wherein PDR-1 and PINK-1 work independently to regulate glial engulfment, and losing both causes strong defects in glial engulfment, as demonstrated by the increased number of fragments for pdr-1; pink-1 relative to wildtype, for animals at sexual maturation (young adult);

FIG. 7 shows pdr-1 mutants result in premature engulfment, as demonstrated by a higher number of engulfed fragments in glia of pdr-1 mutants (right bar) compared to wild type (left bar) at 0 hrs, 16 hrs, and 20 hours during transition to sexually mature adult (post-L4);

FIG. 8 shows a second pdr-1 mutant also results in premature onset of engulfment, as demonstrated by a higher number of engulfed fragments in glia of pdr-1 mutants (right bar) compared to wild type (left bar) at 0 hrs, 16 hrs, and 20 hours during transition to sexually mature adult (post-L4);

FIG. 9 shows pdr-1 mutants have variable engulfment that persists in sexually mature adults, suggesting that steady-state engulfment is not altered and regulation is related to the timing of when the process starts, as demonstrated by increased engulfed fragments in glia of pdr-1 mutant animals (right bars in each set) at day 1 (young) adults but not day 4 adults (nearing end of sexual maturity) compared to wild type (left bars in each set);

FIG. 10 shows disrupting mitophagy does not affect glial engulfment, wherein loss of loss of ucp-4 inhibits mitophagy, but such loss alone does not affect glial engulfment by itself, nor does it affect the pdr-1 phenotype;

FIG. 11A shows PDR-1 is at the glial tip that contacts the AFD-ending which is digested and is not associated with engulfed fragments in the glial cell body, as demonstrated by expression analysis, and thus PDR-1 is at the active site to regulate engulfment and not likely in the endolysosome degradation machinery;

FIG. 11B shows PDR-1 becomes localized into the nucleus by excess CED-10; increased levels of CED-10/Rac1 translocate PDR-1/Parkin to glial nuclei;

FIG. 12 shows different isoforms exist for PDR-1 and that the mutation in gk448 loses isoform B with the Ubl domain, but maintains isoform A with RING2 domain;

FIG. 13A shows the loss of Ubl domain alone is sufficient to cause defects, wherein PDR-1 requires its N-terminal Ubl domain;

FIG. 13B shows the different mutations in pdr-1 and that loss of Ubl domain alone is sufficient to cause defects similar to complete loss of PDR-1, wherein PDR-1 requires its N-terminal Ubl domain;

FIG. 14 shows the PINK-1 kinase domain regulates function, wherein mutations that retain the Ser/Thr kinase domain function of PINK-1 do not show defects like the complete null mutation, as demonstrated by increased engulfed fragments in glia only in the pink-1 (tm1779) null mutant;

FIG. 15A shows PDR-1 acts through JNK signaling in glia engulfment, wherein loss of the JNK kinase KGB-1 abrogates pdr-1 mutant defects, indicating PDR-1 acts with KGB-1 to regulate engulfment in pre-adult animals;

FIG. 15B shows PDR-1 acts through JNK signaling in glia engulfment, wherein loss of the JNK kinase KGB-1 abrogates pdr-1 mutant defects, indicating PDR-1 acts through KGB-1 to regulate engulfment in adult animals;

FIG. 16 shows a JNK inhibitor blocks precocious engulfment of pdr-1, wherein precocious engulfment of a pre-adult is suppressed;

FIG. 17 shows PINK-1 acts through JNK signaling in glia engulfment independent from PDR, where blocking JNK signaling suppresses pink-1 defects, as demonstrated by rescue of increased glial fragments of pink-1 mutants alone by a secondary mutation in kgb-1;

FIG. 18 shows PDR-1 acts through CED-10/Rac1 in glia engulfment, wherein loss of CED-10/Rac1 GTPase mutants can suppress pdr-1 mutant defects;

FIG. 19 shows PDR-1 regulates CED-10/Rac1 levels in glia, wherein null pdr-1 mutants lose CED-10 localization while some alleles have excess, indicating normal levels of CED-10 at the glia tip where engulfment occurs is regulated by PDR-1;

FIG. 20A shows blocking CED-10 alters engulfment defects in adults, as demonstrated by reduction in the number of fragments in glia of pdr-1 mutants with the Rac1 inhibitor;

FIG. 20B shows that a Rac inhibitor drug has pleiotropic defects in engulfment, as seen by longer engulfed glia fragments compared to steady-state wildtype animals when treated with Rac1 inhibitors;

FIG. 21A shows expressing the glia-specific uncoupling factor UCP-4 in glia can suppress engulfment defects of pdr-1 lesion animals, wherein altering mitochondrial uncoupling alone does not affect glial engulfment (ucp-4 mutant), as demonstrated by a reduced number of engulfed fragments in glia in double transgenic animals;

FIG. 21B shows DNP does not suppress engulfment defects, as demonstrated by there being no change in number of engulfed glial fragments in pdr-1 mutant animals;

FIG. 21C shows pruning increase similar to loss of pdr-1, upon treatment with 10 μM of the drug EHOP-016 which inhibits CED-10/Rac1 without losing gene product (left); the pdr-1 defect is also increased (right);

FIG. 22 shows altering glial mitochondria dynamics affects engulfment, wherein miro-1 traffics mitochondria to extremities, and active cells require fzo-1 for mitochondria fusion, as demonstrated by altered number of engulfed AFD neuron fragments in glia of fzo-1 or miro-1 mutant animals;

FIG. 23A shows the drug MG-132 suppresses pdr-1 defects, wherein MG-132 can induce JNK activity and reduce Rac1 activity, as demonstrated by reduction in number of engulfed fragments of neuron within glia of pdr-1 mutant animals upon MG-132 exposure;

FIG. 23B shows the drug rapamycin does not affect pdr-1, as demonstrated by no change in number of engulfed fragments in glia of pdr-1 mutant animals upon rapamycin exposure; rapamycin can induce JNK activity, suggesting that specific JNK activities are differently regulated in glia.

### DETAILED DESCRIPTION

The present disclosure describes a method for regulating glia-directed engulfment and pruning of Neuronal Receptive Endings (NRE) for use in treating neurological diseases such as Parkinson's disease (PD) in a subject, comprising delivering to a subject a therapeutically effective amount of a composition effective in modulating at least one gene and/or protein in a glia cell. In some embodiments, the at least one gene and/or protein is associated with glia-directed engulfment and pruning of Neuronal Receptive Endings (NRE).

Unless specifically defined herein, all terms used herein have the same meaning as they would to one skilled in the art.

As used herein, “or” in the claims is used to mean “and/or” unless explicitly indicated to refer to alternatives only, or the alternatives are mutually exclusive.

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, and to indicate “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

As used herein, “about” refers to an acceptable degree of error for the quantity measured given the nature or precision of the measurements. Typical, exemplary degrees of error are within ±20 percent (%); within ±10%; or within ±5% of a given value or range of values. Any reference to “about X” specifically indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to teach and provide written support for a claim limitation of, e.g., “0.98X.” Alternatively, in biological systems, the term “about” can mean values that are within an order of magnitude, within 5-fold, or within 2-fold of a given value. Numerical quantities given herein are approximate unless stated otherwise, meaning that the term “about” can be inferred when not expressly stated.

As used herein, “subject” encompasses, without limitation, a mammal, which includes all mammals such as humans, non-human primates, dogs, cats, horses, sheep, goats, cows, rabbits, pigs, and rodents. As used herein, the term “subject” and “patient” may be used interchangeably, and mean a mammal in need of treatment, e.g., companion/domestic animals (e.g., dogs, cats, horses, rabbits, and the like), farm animals (e.g., cows, pigs, horses, sheep, goats, and the like) and laboratory animals (e.g., rats, mice, guinea pigs and the like). Typically, the subject is a human in need of treatment.

As used herein, “treating” means the treatment of the disease or condition of interest in a mammal, preferably a human, having the disease or condition of interest, and includes:
(a) preventing the disease or condition from occurring in a mammal, in particular, when such mammal is predisposed to the condition but has not yet been diagnosed as having it;
(b) inhibiting the disease or condition, i.e., arresting the disease or condition development, or delaying the onset of the disease or condition;
(c) relieving (or ameliorating) the disease or condition, i.e., causing regression of the disease or condition; or
(d) relieving (or ameliorating) the symptoms resulting from the disease or condition, e.g., without addressing the underlying disease or condition.

The terms “treating” or ‘treatment” also refer to obtaining a desired pharmacological and/or physiological effect.

As used herein, the term “neurodegenerative disease” refers to a disease or condition in which the function of a subject's nervous system becomes impaired. Examples of neurodegenerative diseases that may be treated with the method described herein are selected from Alzheimer's disease, autism spectrum, leprosy, glioma, Parkinson disease, drug-induced Parkinsonism, Idiopathic Parkinson's disease, Autosomal dominant Parkinson disease, familial type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), and/or Mitochondrial Parkinson's disease.

As used herein, “signaling pathway” refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

As used herein, “therapeutically effective amount” or “effective amount” refers to the amount of a compound of the disclosure that, when administered to a mammal (e.g., a human), is sufficient to effect treatment as defined herein, reduction in symptoms, cure, prevention of onset, or delayed onset, of a disease or condition in the mammal, preferably a human. The amount of a composition of the disclosure which constitutes a “therapeutically effective amount” or an “effective amount” will vary depending on the composition; the condition and its severity; the manner of administration; and the age, weight, and genetics of the mammal to be treated, but can be determined routinely by one of ordinary skill in the art having regard to his own knowledge and to this disclosure.

As used herein, “aberrant” means a glia-directed engulfment and active pruning of NREs which is different from that found in a typical or average organism, such as a human. For example, an aberrant engulfment and active pruning can be an amount or rate which is more than the amount or rate in which engulfment and active pruning typically occur, can be an amount or rate which is less than the amount or rate in which engulfment and active pruning typically occur, can occur at a time which differs from the time in which engulfment and active pruning typically occur, can occur at an age which differs from the age in which engulfment and active pruning typically occur, or it can be a combination of any of the foregoing. Each of the foregoing applies to one or more than one gene, and/or one or more than one protein, wherein each gene or protein can be the same or can be different.

As used herein, “engulfment” means the taking up, consumption, cleavage, digestion, or the like, of the matter (e.g., neuronal receptive ending matter) which was pruned from the neuron.

As used herein, “pruning” means the cleavage, digestion, removal, or the like, wherein the matter being pruned (e.g., neuronal receptive ending matter) is entirely pruned from the neuron, or wherein at least about 90% is pruned from the neuron, at least about 80% is pruned from the neuron, at least about 70% is pruned from the neuron, at least about 60% is pruned from the neuron, at least about 50% is pruned from the neuron, at least about 40% is pruned from the neuron, at least about 30% is pruned from the neuron, at least about 20% is pruned from the neuron, or at least about 10% is pruned from the neuron.

As used herein, “active” pruning means pruning which occurs that is not a part of any phagocytic function of glial cells.

As used herein, “regulating” means adjusting the amount, rate, timing, or stage such as to obtain an amount, rate, timing, or stage which is about the amount, rate, timing, or stage as is typically found in an organism such as a human, or which is the amount, rate, timing, or stage of an average organism, such as a human. The adjusted amount, rate, timing, or stage can vary from the typical or average amount, rate, timing, or stage by up to about 50%, up to about 40%, up to about 30%, up to about 20%, or up to about 10%.

As used herein, “pre-mature” onset means onset of glia-mediated NRE engulfment before the time or life stage in which onset typically occurs in an organism, or occurs in the average organism, such as a human.

As used herein, “delayed” onset means onset of glia-mediated NRE engulfment later than the time or life stage in which onset typically occurs in an organism, or occurs in the average organism, such as a human.

The composition disclosed herein can be a pharmaceutically acceptable salt. A “pharmaceutically acceptable salt” includes both acid and base addition salts.

A pharmaceutically acceptable acid addition salt refers to salts which retain the biological effectiveness and properties of the free bases, which are not biologically, or otherwise, undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A pharmaceutically acceptable base addition salt refers to salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts, and the like. Preferred inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like. Particularly preferred organic bases are isopropylamine, diethylamine, ethanolamine, trimethylamine, dicyclohexylamine, choline, and caffeine.

As used herein, “Parkin” or “PARK2” refers to any native Parkin encoded by the Parkin gene or its ortholog. The term encompasses “full-length,” unprocessed Parkin as well as any form of Parkin that results from processing in the cell, isoforms, and recombinant forms of Parkin. The term also encompasses naturally occurring Parkin and variants of Parkin, e.g., splice variants or allelic variants, functional fragments, isoforms, and homologs, thereof. The term includes Parkin and variants thereof that maintain Parkin activity (e.g., within at least 30%/a, 40%, 50%, 60%, 70%, 80%, 90%, 95%, or 100% activity compared to Parkin). The DNA sequence of an exemplary human Parkin is shown in SEQ ID NO: 24. The RNA sequence of an exemplary C. Elegans PDR-1 is shown in SEQ ID NOs: 1-3.

The term “PINK1,” “PINK,” “PINK-1,” or “PARK8” is used to refer to any native PINK1 encoded by the PINK1 gene or its ortholog. The term encompasses “full-length,” unprocessed PINK1 as well as any form of PINK1 that results from processing in the cell, isoforms and variants of PINK1, e.g., splice variants or allelic variants, functional fragments, and homologs, thereof. The term includes a recombinant or naturally occurring form of PINK1 (e.g. “PTEN induced putative kinase 1”; Entrez Gene 65018, OMIM 608309, UniProtKB Q9BXM7, and/or RefSeq (protein) NP_115785.1). The term includes PINK1 and variants thereof that maintain PINK1 activity (e.g. within at least 30%/a, 40%, 50%, 60%, 70%, 80%, 90%, 95%, or 100% activity compared to PINK1). The DNA sequence of an exemplary human PINK1 is shown in SEQ ID NO: 25. The RNA sequence of an exemplary C. Elegans PINK-1 is shown in SEQ ID NO: 6.

The term “UCP4” or “UCP-4” refers to any native UCP4 encoded by the UCP4 gene or its ortholog. The term encompasses “full-length,” unprocessed UCP4 as well as any form of UCP4 that results from processing in the cell, isoforms and variants of UCP4, e.g., splice variants or allelic variants, functional fragments, and homologs, thereof. The term includes and recombinant or naturally occurring form of UCP4. The term includes UCP4 and variants thereof that maintain UCP4 activity (e.g. within at least 30%/a, 40%, 50%, 60%, 70%, 80%, 90%, 95%, or 100% activity compared to UCP4). The RNA sequence of an exemplary C. Elegans UCP4 is shown in SEQ ID NO: 15. The DNA sequence of an exemplary human UCP4 ortholog, SLC25A7, is shown in SEQ ID NO: 36.

The term “CED-10” refers to any native CED-10 encoded by the CED-10 gene or its ortholog. The term encompasses “full-length,” unprocessed CED-10 as well as any form of CED-10 that results from processing in the cell, isoforms and variants of CED-10, e.g., splice variants or allelic variants, functional fragments, and homologs, thereof. The term includes and recombinant or naturally occurring form of CED-10. The term includes CED-10 and variants thereof that maintain CED-10 activity (e.g. within at least 30%/a, 40%, 50%, 60%, 70%, 80%, 90%, 95%, or 100% activity compared to CED-10). The RNA sequence of an exemplary C. Elegans CED-10 is shown in SEQ ID NOs: 4 and 5. The DNA sequence of an exemplary human CED-10 ortholog, Rac1, is shown in SEQ ID NO: 26.

The term “JNK” refers to any native JNK encoded by the JNK gene or its ortholog. The term encompasses “full-length,” unprocessed JNK as well as any form of JNK that results from processing in the cell, isoforms and variants of JNK, e.g., splice variants or allelic variants, functional fragments, and homologs, thereof. The term includes recombinant or naturally occurring form of JNK. The term includes JNK and variants thereof that maintain JNK activity (e.g. within at least 30%/a, 40%, 50%, 60%, 70%, 80%, 90%, 95%, or 100% activity compared to JNK). The RNA sequence of an exemplary C. Elegans JNK is shown in SEQ ID NOs: 9 and 10. The DNA sequence of an exemplary human JNK ortholog, MAPK8, is shown in SEQ ID NO: 43.

The term “CED-2” refers to any native CrkII encoded by the CRK or CRKL genes or its ortholog. The term encompasses “full-length,” unprocessed CED-2 as well as any form of CED-2 that results from processing in the cell, isoforms and variants of CED-2, e.g., splice variants or allelic variants, functional fragments, and homologs, thereof. The term includes recombinant or naturally occurring form of CED-2. The term includes CED-2 and variants thereof that maintain CED-2 activity (e.g. within at least 30%/a, 40%, 50%, 60%, 70%, 80%, 90%, 95%, or 100% activity compared to CED-2). The RNA sequence of an exemplary C. elegans CED-2 is shown in SEQ ID NOs: 16. The DNA sequence of an exemplary human ortholog, CRK is shown in SEQ ID NO: 29

The term “CED-5” refers to any native DOCK encoded by the CED-5 gene or its ortholog. The term encompasses “full-length,” unprocessed CED-5 as well as any form of CED-5 that results from processing in the cell, isoforms and variants of CED-5, e.g., splice variants or allelic variants, functional fragments, and homologs, thereof. The term includes recombinant or naturally occurring form of CED-5. The term includes CED-5 and variants thereof that maintain CED-5 activity (e.g. within at least 30%/a, 40%, 50%, 60%, 70%, 80%, 90%, 95%, or 100% activity compared to CED-5). The RNA sequence of an exemplary C. elegans CED-5 is shown in SEQ ID NOs: 17. The DNA sequence of an exemplary human ortholog, DOCK1 is shown in SEQ ID NO: 30. Other orthologs include DOCK2, DOCK3, DOCK4, DOCK5.

The term “CED-12” refers to any native CED-12/ELMO encoded by the ELMO genes or its ortholog. The term encompasses “full-length,” unprocessed CED-12 as well as any form of CED-12 that results from processing in the cell, isoforms and variants of CED-12, e.g., splice variants or allelic variants, functional fragments, and homologs, thereof. The term includes recombinant or naturally occurring form of CED-12. The term includes CED-12 and variants thereof that maintain CED-12 activity (e.g. within at least 30%/a, 40%, 50%, 60%, 70%, 80%, 90%, 95%, or 100% activity compared to CED-12). The RNA sequence of an exemplary C. elegans CED-12 is shown in SEQ ID NOs: 18. The DNA sequence of an exemplary human ortholog, ELMO1 is shown in SEQ ID NO: 31. Other orthologs include ELMO2 (SEQ ID NO: 32) and ELMO3 (SEQ ID NO: 33).

The term “TAT-1” refers to any native TAT-1 encoded by the ATP8A gene or its ortholog. The term encompasses “full-length,” unprocessed TAT-1 as well as any form of TAT-1 that results from processing in the cell, isoforms and variants of TAT-1, e.g., splice variants or allelic variants, functional fragments, and homologs, thereof. The term includes recombinant or naturally occurring form of TAT-1. The term includes TAT-1 and variants thereof that maintain TAT-1 activity (e.g. within at least 30%/a, 40%, 50%, 60%, 70%, 80%, 90%, 95%, or 100% activity compared to TAT-1). The RNA sequence of an exemplary C. elegans TAT-1 is shown in SEQ ID NOs: 19, 20, 21. The DNA sequence of an exemplary human ortholog, ATP8A is shown in SEQ ID NOs: 27 and 28.

The term “LRK-1” refers to any native LRRK2 encoded by the LRRK2 gene or its ortholog. The term encompasses “full-length,” unprocessed LRRK2 as well as any form of LRRK2 that results from processing in the cell, isoforms and variants of LRRK2, e.g., splice variants or allelic variants, functional fragments, and homologs, thereof. The term includes recombinant or naturally occurring form of LRRK2. The term includes LRRK2 and variants thereof that maintain LRRK2 activity (e.g. within at least 30%/a, 40%, 50%, 60%, 70%, 80%, 90%, 95%, or 100% activity compared to LRRK2). The DNA sequence of LRRK2, the exemplary human ortholog of C. elegans LRK-1, is shown in SEQ ID NO: 34.

The term “PSR-1” refers to any native JMJD6 encoded by the JMJD6 gene or its ortholog. The term encompasses “full-length,” unprocessed JMJD6 as well as any form of JMJD6 that results from processing in the cell, isoforms and variants of JMJD6, e.g., splice variants or allelic variants, functional fragments, and homologs, thereof. The term includes recombinant or naturally occurring form of JMJD6. The term includes JMJD6 and variants thereof that maintain JMJD6 activity (e.g. within at least 30%/a, 40%, 50%, 60%, 70%, 80%, 90%, 95%, or 100% activity compared to JMJD6). The RNA sequence of an exemplary C. elegans PSR-1 is shown in SEQ ID NOs: 11, 12, 13. The DNA sequence of an exemplary human ortholog, JMJD6 is shown in SEQ ID NO: 42.

The term “DJR-1.2” refers to any native PARK7 encoded by the DJ-1 gene or its ortholog. The term encompasses “full-length,” unprocessed DJ-1 as well as any form of DJ-1 that results from processing in the cell, isoforms and variants of DJ-1, e.g., splice variants or allelic variants, functional fragments, and homologs, thereof. The term includes recombinant or naturally occurring form of DJ-1. The term includes DJ-1 and variants thereof that maintain DJ-1 activity (e.g. within at least 30%/a, 40%, 50%, 60%, 70%, 80%, 90%, 95%, or 100% activity compared to DJ-1). The DNA sequence of DJ-1, the exemplary human ortholog of C. elegans DJR-1.2, is shown in SEQ ID NO: 35.

The term “FZO-1” refers to any native Mitofusin encoded by the MFN-1 and MFN2 genes in humans or their ortholog. The term encompasses “full-length,” unprocessed MFN as well as any form of MFN that results from processing in the cell, isoforms and variants of MFN, e.g., splice variants or allelic variants, functional fragments, and homologs, thereof. The term includes recombinant or naturally occurring form of MFN. The term includes MFN and variants thereof that maintain MFN activity (e.g. within at least 30%/a, 40%, 50%, 60%, 70%, 80%, 90%, 95%, or 100% activity compared to MFN). The DNA sequence of MFN1 and MFN2, the exemplary human orthologs of C. elegans FZO-1, are shown in SEQ ID NO: 37 and SEQ ID NO: 38, respectively.

The term “MIRO-1” refers to any native RHOT encoded by the RHOT1 or RHOT2 genes or their orthologs. The term encompasses “full-length,” unprocessed RHOT as well as any form of RHOT that results from processing in the cell, isoforms and variants of RHOT, e.g., splice variants or allelic variants, functional fragments, and homologs, thereof. The term includes recombinant or naturally occurring form of RHOT. The term includes RHOT and variants thereof that maintain RHOT activity (e.g. within at least 30%/a, 40%, 50%, 60%, 70%, 80%, 90%, 95%, or 100% activity compared to RHOT). The DNA sequence of RHOT1 and RHOT2, the exemplary human orthologs of C. elegans MIRO-1, are shown in SEQ ID NO: 39 and SEQ ID NO: 40, respectively.

A critical function of different glia, including astrocyte glia, microglia, RPE glia-like cells, Schwann cells and recently, oligodendrocytes, is to prune, engulf, or take up fragments of neurons. These fragments may include components of synapses and neuron-receptive endings (NREs). Glia properties and glial engulfment (also called pruning) are altered in many neurological diseases.

The nomenclature of all genes and their human orthologs are as follows: C. elegans PDR-1 (SEQ ID NOs: 1-3) is human Parkin/PARK2 (SEQ ID NO: 24); C. elegans PINK-1 (SEQ ID NO: 6) is human PINK/PARK8 (SEQ ID NO: 25); C. elegans CED-10 (SEQ ID NOs: 4 and 5) is human Rac1 (SEQ ID NO: 26); C. elegans KGB-1 (SEQ ID NO: 7), KGB-2 (SEQ ID NO: 8), and JNK-1 (SEQ ID NOs: 9 and 10) are paralogs of human JNK kinases; C. elegans CED-2 (SEQ ID NO: 16) is human CrkII (SEQ ID NO: 29); C. elegans CED-5 (SEQ ID NO: 17) is human DOCK1 (SEQ ID NO: 30); C. elegans CED-12 (SEQ ID NO: 18) is human ELMO (SEQ ID NOs: 31-33); C. elegans TAT-1 (SEQ ID NO: 19-21) is human ATP8A (SEQ ID NOs: 27 and 28); C. elegans PAT-2 (SEQ ID NO: 14) is human integrin; C. elegans LRK-2 is human LRRK (SEQ ID NO: 34); C. elegans DJR-1.2 is human DJR (SEQ ID NO: 35); C. elegans UCP-4 (SEQ ID NO: 15) is human SLC25A7 (SEQ ID NO: 36); C. elegans FZO-1 is human Mitofusin1 and 2 (SEQ ID NOs: 37 and 38), C. elegans MIRO-1 is human RHOT1 and 2 (SEQ ID NOs: 39 and 40), as shown in Table 1.

TABLE 1
Associated Genes and Identification Numbers

| | | Human | | |
|---|---|---|---|---|
| C. Elegans | SEQ | ortholog/ | SEQ | Database accession |
| (gene name) | ID NO | paralog | ID NO | number |
| PDR-1A | 1 | Parkin/ | 24 | NM_013988.3-gene |
| PDR-1B | 2 | PARK2 | | |
| PDR-1C | 3 | | | |
| CED-10A | 4 | Rac1 | 26 | NM_018890.4-gene |
| CED-10B | 5 | Rac2 | 41 | NM_002872.5-gene |
| PINK-1 | 6 | PINK/ | 25 | NM_032409.3-gene |
| | | PARK8 | | |
| KGB-1 | 7 | MAPK8 | 43 | NM_139049.4-gene |
| KGB-2 | 8 | MAPK9 | 44 | NM_002752.5-gene |
| JNK-1A | 9 | MAPK10 | 45 | NM_138982.4-gene |
| JNK-1B | 10 | | | |
| PSR-1A | 11 | JMJD6 | 42 | NM_001081461.2-gene |
| PSR-1B | 12 | | | |
| PSR-1C | 13 | | | |
| PAT-2 | 14 | integrin | | |
| UCP-4 | 15 | SLC25A7 | 36 | NM_004277.5-gene |
| CED-2 | 16 | CrkII | 29 | NM_005206.5-gene |
| CED-5 | 17 | DOCk1 | 30 | NM_001380.5-gene |
| CED-12 | 18 | ELMO1 | 31 | NM_001206480.2-gene |
| | | ELMO2 | 32 | NM_133171.5-gene |
| | | ELMO3 | 33 | NM_024712.5-gene |
| TAT-1A | 19 | ATP8A | 27 | NM_006095.2-gene and |
| TAT-1B | 20 | | and 28 | NM_016529.6-gene |
| TAT-1C | 21 | | | |
| LRK-1 | | LRRK | 34 | NM_024652.6-gene |
| DJR-1.2 | | DJR | 35 | NM_007262.5-gene |
| FZO-1 | | Mitofusin1 | 37 | NM_033540.3-gene |
| | | Mitofusin2 | 38 | NM_014874.4-gene |
| MIRO-1 | | RHOT1 | 39 | NM_001033566.3-gene |
| | | RHOT2 | 40 | NM_138769.3-gene |

The nervous system contains two major cell types, glia and neurons. Glia closely associate with neurons, reciprocally monitoring and responding to altered activity. Glia-neuron interactions are critical for nervous system development and function.

One mechanism by which glia modulate neural development is by engulfing degenerating neurons, projections, and synaptic material. This has been observed across species (Drosophila, mouse, human, zebrafish) in both the CNS and PNS. Glial engulfment of neuron fragments eliminates exuberant projections in development and removes synapses that fail to adequately mature. In both the Central Nervous System (CNS) and Peripheral Nervous System (PNS) of adult animals, multiple glia types across various species clear degenerating neural debris after injury.

The regulatory logic by which glia regulate engulfment of neuron fragments remains poorly defined. In vitro and in vivo models ranging from Caenorhabditis elegans to mammals have been used to explore glial phagocytic activity, show striking molecular similarities across these systems and species. This alludes to evolutionary conservation of glial function.

C. elegans AMsh glial cells engulf Neuronal Receptive Ending (NRE) fragments of the neuron of the animal. Thus, glial function is conserved in the nematode and across sense-organ glia. Further, engulfment requires the phospholipid transporter TAT-1/ATP8A, α-integrin PAT-2, and glial phosphatidylserine receptor PSR-1. PSR-1 engages a conserved ternary GEF complex (CED-2/CrkII, CED-5/DOCK1, CED-12/ELMO1) to activate CED-10/Rac1 GTPase. The actin remodeling factor WSP-1/nWASp, a known effector of CED-10, acts in glia to regulate engulfment. Glial engulfment rates are regulated by temperature and track AFD neuron activity. Glial CED-10/Rac1 acts downstream of neuron activity, and CED-10 expression levels dictate NRE engulfment rates. Regulation of CED-10/Rac1 levels regulate the rate of glial pruning.

Genes that interact with CED-10/Rac1 include PDR-1 in glial pruning. PDR-1/Parkin is linked to disease, and LRK-2, PINK-1 and DJR-1.2 also have been implicated in neurodegenerative disease. These genes are linked to neuronal mitophagy, and regulators of mitochondria include UCP-4, FZO-1, and MIRO-1.

Perturbation of glial engulfment leads to defects in AFD-NRE shape. The present disclosure shows that glia actively regulate engulfment and glia-dependent engulfment of neuron endings can be dynamically regulated. Thus, glia in other organisms may similarly deploy regulated phagocytosis to tune sensory NREs and synapses, and dynamically modulate adult animal behaviors.

In one aspect, the present disclosure provides a method for regulating an aberrant glia-directed engulfment and active pruning of Neuronal Receptive Endings (NRE), wherein the method comprises contacting a neuronal and/or a glia cell with an effective amount of a composition effective in modulating at least one gene and/or protein associated with glia-directed engulfment and active pruning of Neuronal Receptive Endings (NRE).

In some embodiments, the at least one gene and/or protein associated with glia-directed engulfment and active pruning of Neuronal Receptive Endings (NRE) comprises a gene in, and/or a protein expressed by, a neuronal cell, a glia cell, or a combination thereof.

In some embodiments, the at least one gene and/or protein associated with glia-directed engulfment and active pruning of Neuronal Receptive Endings (NRE) comprises a gene in, and/or a protein expressed by a glia cell.

In some embodiments, the at least one gene and/or protein associated with glia-directed engulfment and active pruning of Neuronal Receptive Endings (NRE) is selected from: P4-type ATPase/ATP8A, phosphatidyl serine receptor (PSR-1), PAT-2/α-integrin, CED2/CrkII, CED5/DOCK1, CED-12/ELMO, PDR-1/PARKIN (E3 ubiquitin ligase), PTEN-induced putative kinase 1 (PINK1) PINK1/PARK6 (mitochondrial serine threonine kinase), UCP-4/SLC25A27, DJR-1.2/DJR, LRK-1/LRRK2, KGB-1/JNK, KGB-2/JNK, CED-10/Rac1, variants, isoforms thereof, and combinations thereof. Where the name of the protein is indicated, this disclosure includes the gene encoding the protein as named and a homolog or ortholog thereof. Thus, in some embodiments, the at least one gene associated with glia-directed engulfment and active pruning of Neuronal Receptive Endings (NRE) is selected from: P4-type ATPase/ATP8A, phosphatidyl serine receptor (PSR-1), PAT-2/α-integrin, CED2/CrkII, CED5/DOCK1, CED-12/ELMO, PDR-1/PARKIN (E3 ubiquitin ligase), PTEN-induced putative kinase 1 PINK1/PARK6 (mitochondrial serine threonine kinase), UCP-4/SLC25A27, DJR-1.2/DJR, LRK-1/LRRK2, KGB-1/JNK, KGB-2/JNK, CED-10/Rac1, homolog or an ortholog thereof.

In some embodiments, the at least one gene and/or protein associated with glia-directed engulfment and active pruning of Neuronal Receptive Endings (NRE) is selected from: CED2/CrkII, PDR-1/PARKIN, PTEN-induced putative kinase 1 (PINK1)/PINK/PARK6, UCP-4/SLC25A27, DJR-1.2/DJR, LRK-1/LRRK2, KGB-1/JNK, KGB-2/JNK, CED-10/Rac1, variants, isoforms thereof, and combinations thereof.

The compositions contemplated by the present disclosure may be effective in modulating the at least one gene, and/or the protein encoded by the at least one gene, associated with glia-directed engulfment and active pruning of Neuronal Receptive Endings (NRE). The compositions disclosed herein may be effective in modulating the at least one protein, an isoform, or a variant thereof associated with glia-directed engulfment and active pruning of Neuronal Receptive Endings (NRE).

In some embodiments, the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof.

For example, the present disclosure contemplates compositions effective in regulating the expression of the at least one gene, homolog, or ortholog thereof by (i) modulating the gene at the transcriptional level, for e.g., including but not limited to, by affecting RNA polymerase activity or access to the gene promoter region; (ii) affecting post-transcriptional modulation of the expression of the gene, for e.g., including but not limited to, affecting processes such as RNA splicing, editing, or stability; (iii) affecting epigenetic modulation of gene expression, including but not limited to, such as DNA methylation or histone modification; (iv) inhibiting the expression of the gene, for e.g., including but not limited to, a small RNA molecules (RNAi) composition targeting the transcript of the at least one gene; and/or (iv) precise modification of gene sequence by deleting, or replacing gene segments by gene editing technologies like CRISPR-Cas9.

In certain embodiments, the modulation of the at least one gene encompasses introduction of the at least one gene in form of a transgene in a neuronal and/or glial cell of an organism or a subject, which may be homologous or heterologous to the organism or the subject, using at least one expression construct comprising the transgene. In an embodiment, the modulation of the at least one gene comprises introduction of the at least one gene in the form of a transgene in a glial cell of the organism or the subject, using at least one expression construct comprising the transgene. An “expression construct” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide encoded by the transgene in a host cell or organism. Standard techniques which are, in particular, especially suited for the introduction of a gene in the cells of an organism, are available and known to the skilled person for transforming or transfecting an organism's cells with constructs which contain the relevant transgenes.

One skilled in the art may refer to general reference texts for detailed descriptions of known techniques discussed herein or equivalent techniques. These texts include Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999). These texts can, of course, also be referred to in making or using an aspect of the invention.

In some embodiments, modulating the at least one gene includes but is not limited to increasing transcription of the at least one gene, decreasing transcription of the at least one gene, inhibiting expression of the at least one gene, or activating expression of the at least one gene expression.

Modulating the expression of the at least one protein comprises effecting an increased expression of the protein or effecting a decreased expression of the protein. Modulating the expression of the at least one protein comprises activating the expression of the protein or inhibiting/blocking the expression of the protein. Nonlimiting exemplary compositions effective in modulating expression of a protein include antisense oligonucleotides and short interfering RNAs (siRNAs) and other inhibitory RNAs specific for the at least one protein. Modulating the activity and/or localization of the at least one protein comprises increasing the activity of the protein or decreasing the activity of the protein. In an exemplary embodiment, modification of protein activity and/or localization comprises changing the actual work done by the protein in a cell. Optionally, as noted herein, the modification of protein activity and/or localization, is for example, by modification of phosphorylation. In some embodiments, the modification of protein activity and/or localization comprises activation and/or inhibition of at least one signaling pathway. In some embodiments, the modulation of the activity and/or localization of the at least one protein comprises modulating an upstream regulator of the activity of the at least one protein in at least one signaling pathway. In some embodiments, the modulation of the activity and/or localization of at least one protein may comprise modulating a downstream effector of the protein in at least one signaling pathway. Nonlimiting exemplary compositions effective in modulating protein activity and/or localization of the at least one protein includes nucleic acids, peptides, peptidomimetics, small molecules, macrolides, anthrapyrazolones, proteins, monoclonal antibodies, pyrimidine diamines, catechols, pharmaceutically acceptable salts thereof, or a combination thereof, specific for the at least one protein.

In some embodiments, the compositions disclosed herein are capable of blocking, neutralizing, inhibiting, abrogating, reducing and/or interfering with one or more of the activities of, and/or reducing the expression of, the at least one protein (or the expression of nucleic acids encoding the at least one protein). In some embodiments, the composition may decrease the activity and/or expression of the at least one protein by at least 10%, 15%, 20%, 25%, 30%, 35%, 40%, 45%, 50%, 55%, 60%, 65%, 70%, 75%, 80%, 85%, 90%, or even 100% decrease, as compared to the expression and/or activity of the at least one protein that is untreated by the composition.

In some embodiments, the compositions disclosed herein are capable of activating, increasing and/or enhancing one or more of the activities of, and/or increasing the expression of, the at least one protein (or the expression of nucleic acids encoding the at least one protein). In some embodiments, the composition may increase the activity and/or expression of the at least one protein by at least 10%, 15%, 20%, 25%, 30%, 35%, 40%, 45%, 50%, 55%, 60%, 65%, 70%, 75%, 80%, 85%, 90%, or even 100% increase, as compared to the expression and/or activity of the at least one protein that is untreated by the composition.

Each of the foregoing methods of modulation can apply to each of the disclosed genes, variants, or orthologs thereof, each of the disclosed proteins, isoforms, mutants, or variants thereof, or a combination thereof. In the disclosed method for regulating aberrant glia-directed engulfment and active pruning of NREs, one, or more than one, gene and/or protein can be modulated in the same or a different way. For example, one or more proteins are activated while a different one or more proteins are inhibited, and yet other one or more different proteins are unaffected; one or more genes are upregulated while a different one or more genes are downregulated, and yet other one or more genes are unaffected; two or more different proteins are activated; two or more different proteins are inhibited; two or more different genes are downregulated; two or more different genes are upregulated; one or more proteins are inhibited and one or more genes are downregulated; one or more proteins are inhibited and one or more genes are upregulated; one or more proteins are activated and one or more genes are upregulated; one or more proteins are activated and one or more genes are downregulated.

In some embodiments, the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof, in a neuronal cell.

In some embodiments, the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof, in a glia cell.

In some embodiments, the composition is selected from a JNK regulator or effector, a Rac1 regulator or effector, a PDR-1/PARKIN regulator or effector, a CED-10 regulator or effector, a PINK regulator or effector, a LRRK regulator or effector, a DJR-1.2 regulator or effector, a KGB-1 regulator or effector, a SLC25A27 regulator or effector, an ATP9A regulator or effector, a CrkII regulator or effector, a DOCK1 regulator or effector, a ELMO regulator or effector, a ATPI3A2 regulator or effector, and combinations thereof.

In some embodiments, the composition comprises a CED-10/Rac1 regulator or effector, a PDR-1/PARKIN regulator or effector, a PINK1/PINK regulator or effector, a LRK-1/LRRK regulator or effector, a DJR-1.2/DJR regulator or effector, a KGB-1/JNK regulator or effector, a KGB-2/JNK regulator or effector, a UCP-4/SLC25A27 regulator or effector, a CED-2/CrkII regulator or effector, a CED-5/DOCK1 regulator or effector, a CED-12/ELMO regulator or effector, and combinations thereof.

In some embodiments, the composition is selected from a nucleic acid, a peptide, a peptidomimetic, a small molecule, a macrolide, an anthrapyrazolone, a protein, a monoclonal antibody, a pyrimidine diamine, a catechol, a pharmaceutically acceptable salt thereof, or a combination thereof.

In some embodiments disclosed herein, the composition is a polynucleic acid comprising DNA or RNA nucleotides.

In some embodiments disclosed herein, the composition is a peptide, wherein the peptide comprises a strand of naturally occurring amino acids, unnatural amino acids, amino acids which have been modified, or a combination thereof. In some embodiments disclosed herein, the composition is a peptidomimetic, wherein the composition mimics a natural or unnatural peptide in sequence or 3-dimensional space. In some embodiments, for peptides or peptidomimetics, one or more amino acids can comprise D- and/or L-stereochemistry; the amino acids can be modified by post-translational modification; and/or the amino acids can be modified prior to administering, such as by methylation, acetylation, acylation, oxidation, phosphorylation, and/or glycosylation. For example, the peptide or peptidomimetic can be MG-132, having the structure:

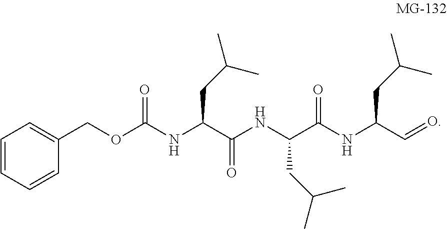 [SMILES: CC(C)C[C@@H](C=O)NC(=O)[C@H](CC(C)C)NC(=O)[C@H](CC(C)C)NC(=O)OCc1ccccc1]

In some embodiments disclosed herein, the composition is a macrolide. For example, the macrolide is rapamycin, having the structure:

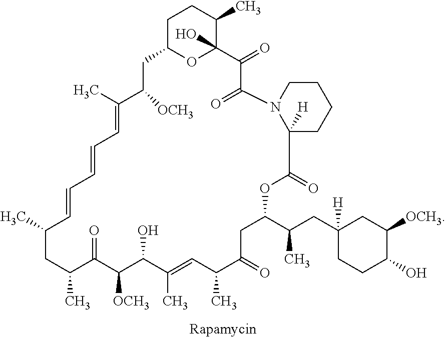

In some embodiments disclosed herein, the composition is an anthrapyrazolone. For example, the anthrapyrazolone is Sp600125, having the structure:

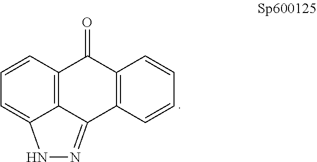 [SMILES: O=C1c2ccccc2-c2n[nH]c3cccc1c23]

In some embodiments disclosed herein, the composition is a pyrimidinediamine. For example, the pyrimidinediamine is EHOP-016, having the structure:

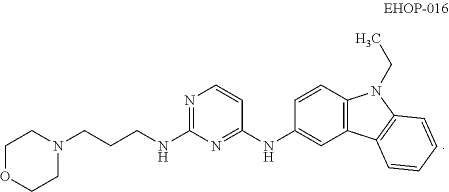 [SMILES: CCn1c2ccccc2c2cc(Nc3ccnc(NCCCN4CCOCC4)n3)ccc21]

In some embodiments disclosed herein, the composition is a catechol. For example, the catechol is apocynin, or acetovanillone, having the structure:

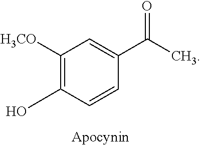 [SMILES: COc1cc(C(C)=O)ccc1O]

In some embodiments disclosed herein, the composition is a small molecule. A small molecule is defined as a molecule having a molecular weight of less than about 2500 Da, of less than about 2000 Da, of less than about 1500 Da, of less than about 1000 Da, of less than about 750 Da, of less than about 500 Da, or of less than about 350 Da.

The small molecule can be MG-132 having the structure as disclosed above. The small molecule can be rapamycin, having the structure as disclosed above. The small molecule can be Sp600125, having the structure as disclosed above. The small molecule can be EHOP-016, having the structure as disclosed above. The small molecule can be apocynin, having the structure as disclosed above.

In some embodiments the composition comprises EHOP-016, Sp600125, MG-132, rapamycin, apocynin, a pharmaceutically acceptable salt thereof, or a combination thereof.

In some embodiments the composition comprises EHOP-016, Sp600125, MG-132, rapamycin, apocynin, a derivative thereof, a pharmaceutically acceptable salt thereof, or a combination thereof.

In some embodiments disclosed herein, the composition is a protein associated with glia-directed engulfment and active pruning of Neuronal Receptive Endings (NRE). In an embodiment, the protein is selected from PDR-1/PARKIN (E3 ubiquitin ligase), PTEN-induced putative kinase 1 (PINK1) PINK1/PARK6 (mitochondrial serine threonine kinase), UCP-4/SLC25A27, DJR-1.2/DJR, LRK-1/LRRK2, KGB-1/JNK, KGB-2/JNK, isoforms, variants, and combinations thereof.

In some embodiments, the composition comprises anti-sense oligonucleotides, small interfering RNAs (siRNAs), short hairpin RNAs (shRNAs), peptide nucleic acids (PNAs), or ribozymes targeting the transcript of the at least one protein associated with glia-directed engulfment and active pruning of Neuronal Receptive Endings (NRE). In some embodiments, the at least one protein is selected from PDR-1/PARKIN (E3 ubiquitin ligase), PTEN-induced putative kinase 1 PINK1/PARK6 (mitochondrial serine threonine kinase), UCP-4/SLC25A27, DJR-1.2/DJR, LRK-1/LRRK2, KGB-1/JNK, KGB-2/JNK and combinations thereof.

In some embodiments, the composition further comprises a pharmaceutically acceptable carrier or excipient, to form a pharmaceutical composition. A “pharmaceutical composition” refers to a formulation of a compound of the disclosure and a medium generally accepted in the art for the delivery of the compound to mammals, e.g., humans. Such a medium includes all pharmaceutically acceptable carriers, diluents, or excipients thereof.

In some embodiments, the method disclosed herein further comprises an agent for enhanced delivery of the composition to the neuronal and/or glia cell. For example, the agent is selected from a targeting ligand, a lipid-based nanoparticle, a polymeric nanoparticle, a cationic polymer, a cell-penetrating peptide, a viral vector, an exosome, and a synthetic nanovesicle. Such agents enhance delivery by increasing the amount of the composition which contacts the neuronal and/or glial cell.

In some embodiments, the composition is effective in editing the gene of the at least one protein associated with glia-directed engulfment and active pruning of Neuronal Receptive Endings (NRE), and wherein the composition comprises: a guide RNA specifically designed to guide a CRISPR/Cas9 complex to the gene of the at least one protein associated with glia-directed engulfment and active pruning of Neuronal Receptive Endings (NRE) and a Cas9 protein or a nucleic acid encoding the Cas9 protein. In some embodiments, the gene is selected from PDR-1/PARKIN (E3 ubiquitin ligase), PTEN-induced putative kinase 1 (PINK1) PINK1/PARK6 (mitochondrial serine threonine kinase), UCP-4/SLC25A27, DJR-1.2/DJR, LRK-1/LRRK2, KGB-1/JNK, KGB-2/JNK and combinations thereof.

In some embodiments, the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof, in a neuronal cell.

In some embodiments the composition modulates a protein in a neuronal cell, the protein comprises P4-type ATPase/ATP8A, a variant or an isoform thereof.

In some embodiments, the at least one gene and/or protein is selected from PSR-1, PAT-2/α-integrin, PINK-1/PINK, PDR-1/PARKIN, LRK-1/LRRK2, DJR-1.2/DJR, CED-10/RAC1, KGB-1/JNK, KGB-2/JNK, UCP-4, a mutant thereof, an isoform thereof, and combinations thereof.

In some embodiments, the at least one gene and/or protein is selected from PINK-1/PINK, PDR-1/PARKIN, CED-10/RAC1, KGB-1/JNK, KGB-2/JNK, UCP-4/SLC25A27, a mutant thereof, an isoform thereof, and combinations thereof.

In some embodiments, the at least one gene and/or protein is PDR-1/PARKIN, a truncated PDR-1/PARKIN, or isoforms thereof.

In some embodiments, the truncated PDR-1/PARKIN comprises a PDR-1/PARKIN protein lacking a Ubl domain.

In some embodiments, the composition is effective in restoring expression of a PDR-1/PARKIN comprising the Ubl domain in the glial cell.

In some embodiments, the composition comprises a modulator of the activity of Janus N-terminal Kinase (JNK) or an isoform thereof, and wherein the JNK or the isoform thereof is a downstream effector of PDR-1/PARKIN.

In some embodiments, the composition is effective in modulating the expression levels, localization, or the activity of Rac1 GTPase (CED-10/RAC1), and wherein the Rac1 GTPase (CED-10/RAC1) is acting with PDR-1/PARKIN.

In some embodiments, the composition is effective in modulating the expression of the mitochondrial uncoupling protein UCP-4/SLC25A7 in the glia cell, wherein the composition comprises an expression vector encoding UCP-4/SLC25A7 or a modulator thereof, and wherein the composition is effective in modulating PDR-1/PARKIN.

In some embodiments, the method is effective in modulating the timing of the glia-mediated NRE engulfment, and wherein the method is effective in preventing pre-mature onset of glia-mediated NRE engulfment, or wherein the method is effective in preventing delayed onset of glia-mediated NRE engulfment.

In some embodiments, the at least one gene and/or protein is PINK-1/PINK, a mutated PINK-1/PINK protein, or a combination thereof.

In some embodiments, the composition is effective in restoring a serine/threonine kinase function of PINK-1 protein.

In some embodiments, the composition comprises a regulator of the activity of Janus N-terminal Kinase (JNK), or an isoform or ortholog thereof, and wherein the Janus N-terminal Kinase (JNK), or the isoform or ortholog thereof, is a modulator of PINK-1.

In some embodiments, the composition is effective in regulating expression levels, localization, or activity of Rac1 GTPase (CED-10/RAC1), and wherein Rac1 GTPase (CED-10/RAC1) is acting with PINK-1.

In some embodiments, the method is effective in regulating neuronal function, neuronal shape, an alteration in the rate of glia-directed engulfment and active pruning of Neuronal Receptive Endings (NRE), timing of glia-directed engulfment and active pruning of Neuronal Receptive Endings (NRE), or a combination thereof.

In some embodiments, the method is effective in treating a neurodegenerative disorder or disease in a subject in need thereof. For example, the neurodegenerative disorder or disease is selected from Parkinson's Disease (PD), Alzheimer's disease, Autism Spectrum Disorders, leprosy, and gliomas. In some embodiments, the neurodegenerative disorder or disease is Parkinson's Disease (PD).

In the embodiments disclosed herein, the subject is a mammal. In an exemplary embodiment, the subject is human, and the composition is effective in modulating at least one gene and/or protein encoded by the gene associated with glia-directed engulfment and active pruning of Neuronal Receptive Endings (NRE). In some embodiments, the at least one gene is the human ortholog of genes selected from: P4-type ATPase/ATP8A, phosphatidyl serine receptor (PSR-1), PAT-2/α-integrin, CED2/CrkII, CED5/DOCK1, CED-12/ELMO, PDR-1/PARKIN (E3 ubiquitin ligase), PTEN-induced putative kinase 1 (PINK1) PINK1/PARK6 (mitochondrial serine threonine kinase), UCP-4/SLC25A27, DJR-1.2/DJR, LRK-1/LRRK2, KGB-1/JNK, KGB-2/JNK, CED-10/Rac1, and combinations thereof.

In some embodiments, the at least one gene is the human ortholog of genes selected from: PDR-1/PARKIN, PTEN-induced putative kinase 1 (PINK1) PINK1/PARK6, UCP-4/SLC25A27, DJR-1.2/DJR, LRK-1/LRRK2, KGB-1/JNK, KGB-2/JNK, CED-10/Rac1, and combinations thereof.

In another aspect, the disclosure provides a method of treating a neurodegenerative disease in a subject in need thereof, the method comprising administering to the subject an effective amount of a therapeutic composition effective in modulating at least one gene and/or protein associated with glia-directed active pruning of Neuronal Receptive Endings (NRE).

In some embodiments, the therapeutic composition comprises the composition or pharmaceutical composition as disclosed herein.

The compositions disclosed herein can be administered alone or can be co-administered to the subject. Co-administration is meant to include simultaneous or sequential administration of the composition in combination with another substance, e.g., a composition as disclosed herein, another active substance, a carrier, a diluent, a stabilizing agent, an emulsifier, a vehicle, and the like. Thus, the compositions can also be combined, when desired, with other active substances (e.g., at least one other therapeutic agent or composition), an agent to reduce metabolic degradation, an agent to increase stability, an agent to enhance delivery (e.g. a liquid medium), or components to provide sustained release and/or comfort. In an embodiment, the compositions disclosed herein are administered with at least one other active agent known to be useful in treating a neurodegenerative disease (e.g., Parkinson's disease, such as levodopa, dopamine agonists (e.g., bromocriptine, pergolide, pramipexole, ropinirole, piribedil, cabergoline, apomorphine, lisuride), MAO-B inhibitors (e.g. selegiline or rasagiline), amantadine, anticholinergics, antipsychotics (e.g. clozapine), cholinesterase inhibitors, modafinil, or non-steroidal anti-inflammatory drugs).

In some embodiments, co-administration includes administering a second active agent within 0.5, 1, 2, 4, 6, 8, 10, 12, 16, 20, or 24 hours, or 2 days, 3 days, 4 days, 7 days, 14 days, 30 days, 60 days, or 90 days of administering the first active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents/compositions. In other embodiments, the active agents or compositions can be formulated separately and administered simultaneously, approximately simultaneously, or separately. In some embodiments, the active agents and/or compositions may be linked or conjugated to one another.

The compositions of the present disclosure can also be delivered as microspheres for slow release in the body. For example, microspheres can be administered by the intradermal injection of composition-containing microspheres, which slowly release subcutaneously (see Rao, J. Biomater Sci. Polym. Ed. 7:623-645, 1995); as biodegradable and injectable gel formulations (see, e.g., Gao Pharm. Res. 12:857-863, 1995); or as microspheres for oral administration (see, e.g., Eyles, J. Pharm. Pharmacol. 49:669-674, 1997). In some embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, the delivery of the compositions of the present disclosure can be focused to the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Administering a composition as described herein, or a pharmaceutically acceptable salt thereof, to a subject comprises any suitable delivery method. Administering a composition described herein, or a pharmaceutically acceptable salt thereof, to a subject includes administering the composition described herein, or the pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the subject, mammal, or human.

Thus, a composition, or pharmaceutically acceptable salt thereof, as described herein, may be systemically administered, e.g., orally, in combination with a pharmaceutically acceptable vehicle or carrier such as an inert diluent or an assimilable edible carrier. The composition may be enclosed in hard or soft shell gelatin capsules, may be compressed into tablets, or may be incorporated directly with the food of the subject's diet. For oral therapeutic administration, the composition or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1%, 1%, 2%, 5%, 10%, 15%, 20%, 25%, 30%, 40%, 50%, 60%, 70%, 80%, or 90% of active compound. The percentage of the compositions and preparations may be varied based on the activity of the composition; the age, size, weight, health of the subject; and the mode of administration. The percentage of the compositions and preparations may be between about 2 to about 60% of the weight of a given unit dosage form. The amount of active compound in such therapeutically useful compositions can be such that an effective dosage level will be obtained.

The disclosed method can include a kit comprising a composition or pharmaceutically acceptable salt thereof as described herein and instructional material which can describe administering the composition or pharmaceutically acceptable salt thereof as described herein to a cell or a subject.

In other aspects, the disclosure provides a method for treating Parkinson's Disease in a subject in need thereof, the method comprising administering to the subject a therapeutically effective amount of a composition effective in modulating at least one gene and/or protein associated with Parkinson's Disease (PD), wherein the gene and/or protein is expressed in a glia cell. In some embodiments, the at least one gene and/or protein is associated with glia-directed engulfment and active pruning of Neuronal Receptive Endings (NRE). In some embodiments, the gene and/or protein is selected from PINK-1/PINK, PDR-1/PARKIN, LRK-1/LRRK2, DJR-1.2/DJR, CED-10/Rac1, KGB/JNK, UCP-4, SLC25A7, a variant, an isoform thereof, and combinations thereof.

In some embodiments, the PD is associated with a loss of function mutation of the at least one gene, wherein the loss of function mutation is homozygous or heterozygous. In some embodiments, the PD is associated with: (i) an increased function of the at least one gene and/or protein, (ii) a decreased function of the at least one gene and/or protein, or (iii) a combination of decreased function of the at least one gene and/or protein and an increased function of a different one or more gene and/or protein.

In some embodiments, the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof.

In some embodiments, the composition is effective in modulating the activity of the at least one protein.

In some embodiments, the composition is effective in modulating at least one gene encoding the at least one protein.

In yet another aspect, the disclosure provides a method for the treatment of Parkinson's disease comprising administering to a subject in need thereof a composition effective in modulating the activity of Janus N-terminal Kinase (JNK) or an isoform thereof, wherein the composition is targeted for delivery to glia cells, and wherein the subject harbors a loss of function gene mutation in PARKIN or PINK1. In some embodiments, the loss of function gene mutation is heterozygous. In some embodiments, the loss of function gene mutation is homozygous. In some embodiments, the composition is selected from an siRNA directed against JNK or an isoform thereof, an artificial microRNA directed against JNK or an isoform thereof, an antisense molecule directed against JNK or an isoform thereof, a ribozyme directed against JNK, a peptide directed against JNK, a peptidomimetic directed against JNK or a small molecule directed against JNK. In some embodiments, the composition further comprises an agent for enhanced and/or targeted delivery of the composition to the glia. Nonlimiting exemplary agents include a targeting ligand specific for a glial cell, a lipid-based nanoparticle, a polymeric nanoparticle, a cationic polymer, cell-penetrating peptide, viral vector, an exosome, and a synthetic nanovesicle, and combinations thereof.

In a further embodiment, the disclosure provides a method for the treatment of Parkinson's disease comprising administering to the subject in need thereof a composition effective in modulating CED-10/Rac-1 or an isoform thereof, wherein the composition is targeted for delivery to the glia, and wherein the subject harbors a loss of function gene mutation in PARKIN or PINK1. In some embodiments, the loss of function mutation is homozygous or heterozygous.

In some embodiments, modulating the CED-10/Rac-1 or an isoform thereof comprises modulating the expression levels, localization, or the activity of Rac1 GTPase (CED-10/RAC1). In some embodiments, the composition further comprises an agent for enhanced and/or targeted delivery of the composition to the glia.

### EXAMPLES

The following examples are set forth to provide those of ordinary skill in the art with a complete disclosure and description of how to make and use the present invention. The following examples are not intended to limit the scope of the invention, nor are they intended to represent the entire scope of experiments performed.

### Example 1

### General Materials and Methods

### Strains

Strains were sourced from (a) the CGC, (b) the International C. elegans Gene Knockout Consortium (C. elegans Gene Knockout Facility at the Oklahoma Medical Research Foundation and the C. elegans Reverse Genetics Core Facility at the University of British Columbia), and (c) National BioResource Project (NBRP), Japan.

### Plasmids

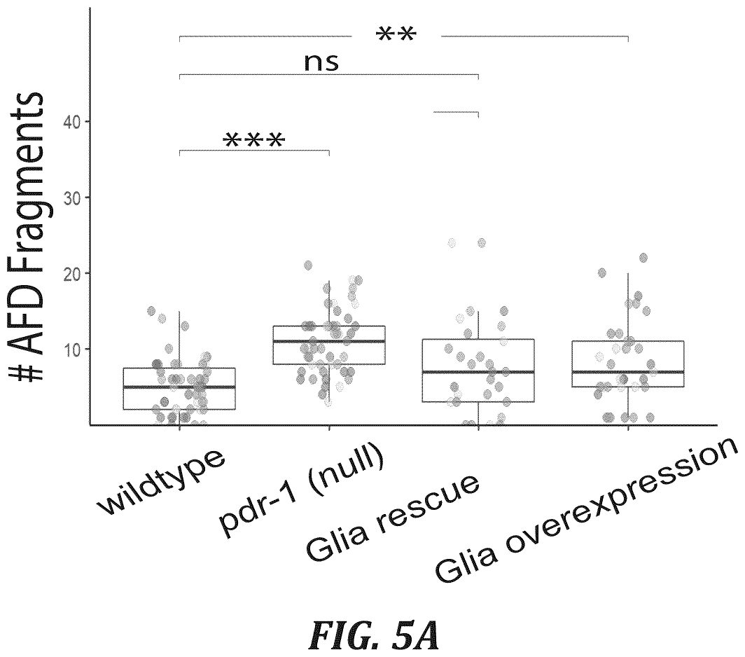

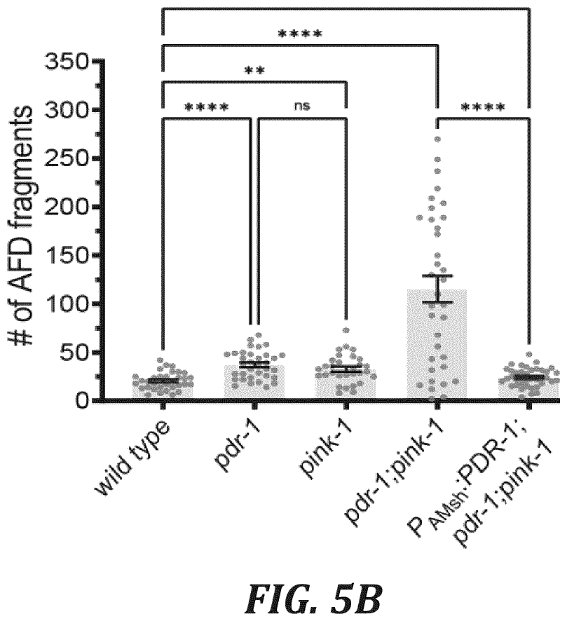

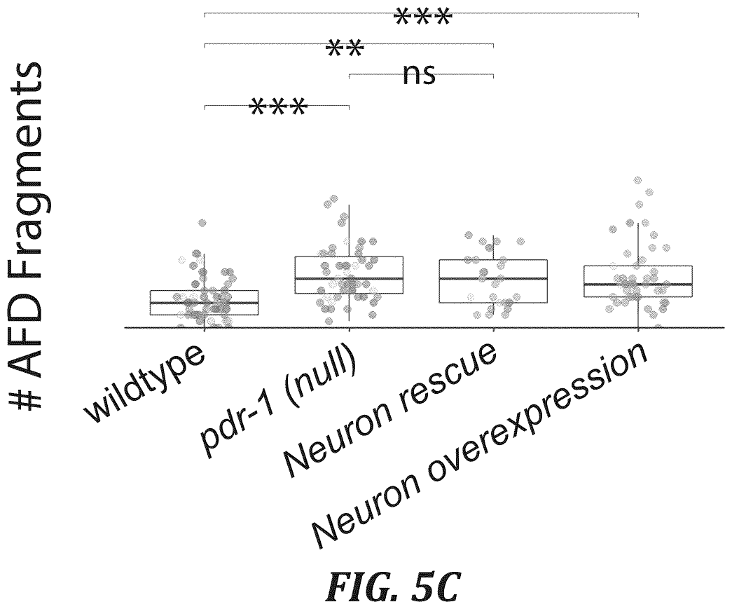

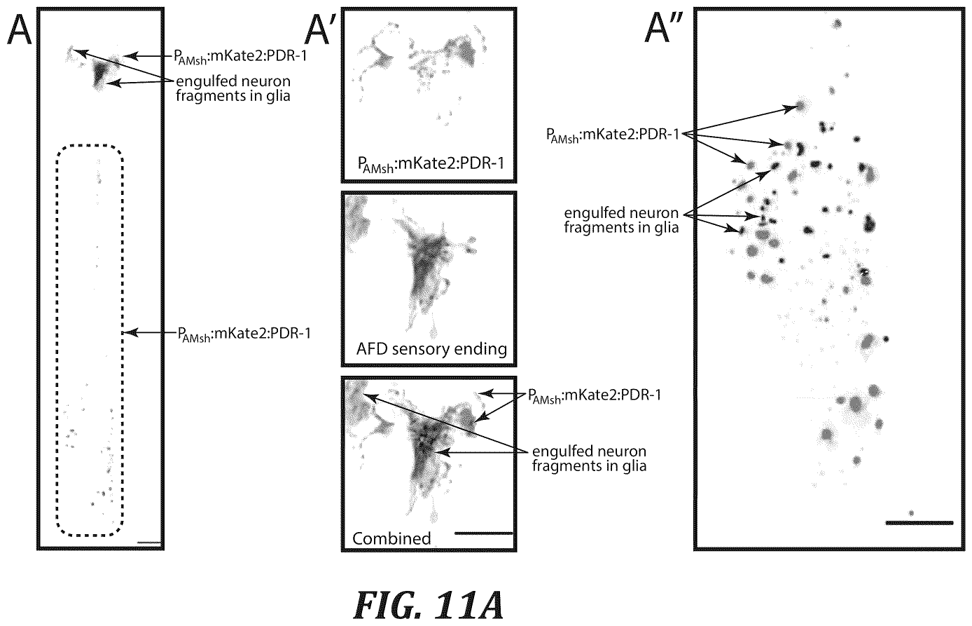

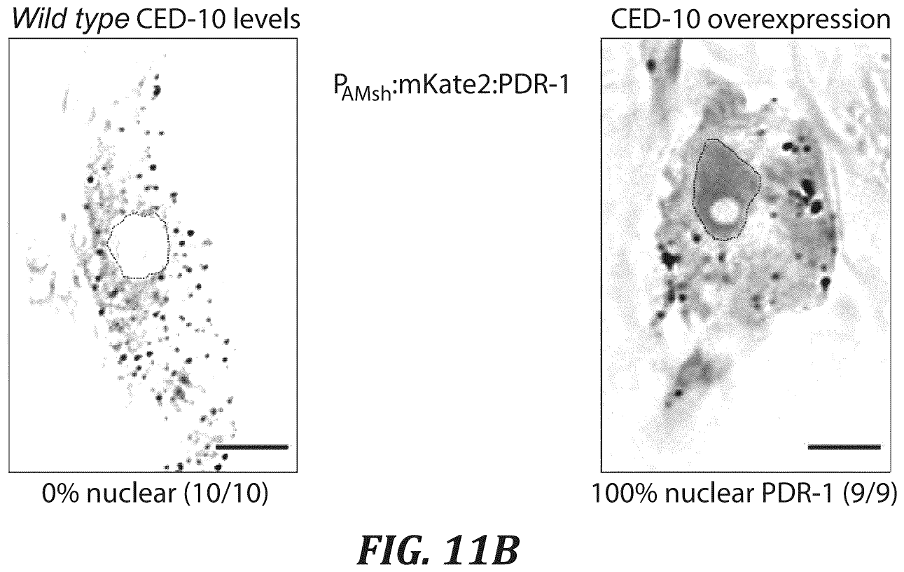

PDR-1/PARK constructs: cDNA for pdr-1 isoform A was isolated via PCR from a mixed stage C. elegans cDNA librarypAS. The cDNA was inserted into the parent vector pAS464 using Gibson Assembly® to generate ASJ187/pSAR47 (P_{F53F4.13}:mKate2:PDR-1) (SEQ ID NO: 56). (FIG. 11). Amplified pdr-1 isoform A was also inserted into the parent vector pAS322 using Xma1/Nhe1 to generate ASJ72/pSAR19 (P_{srtx-1-b}:PDR-1:SL2:mCherry) (SEQ ID NO: 55) and ASJ55/pSAR17 ((FIG. 5). Human PARK2 sequences were inserted into ASJ465 using Xma/Nhe to generate ASJ467/pET7 (P_{F53F4.13}:hPARK2:SL2:mCherry.

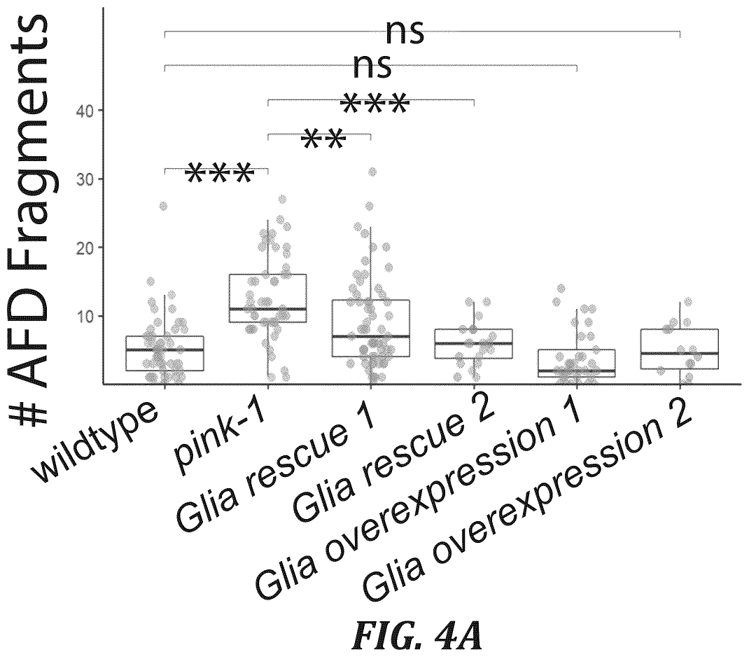

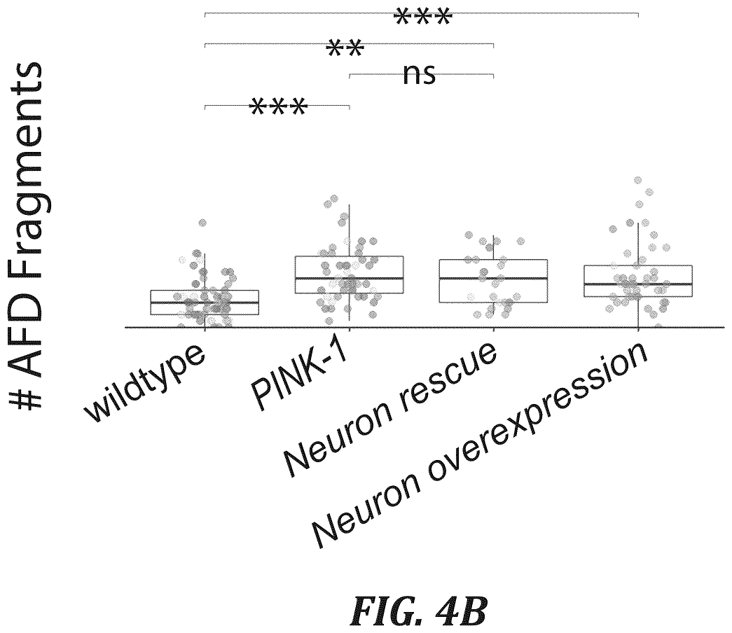

PINK-1 constructs: cDNA for pink-1 was PCR amplified from a mixed-stage C. elegans cDNA library and cloned into pAS548 (P_{F53F4.13}:SL2:mKate2) using Xma1/Xba1 sites to generate ASJ333/pET2 (P_{F53F4.13}:PINK-1:SL2:mKate2) (SEQ ID NO: 48). Amplified pink-1 was also cloned in ASJ40/pMP7 (P_{srtx-1-b}:PINK-1:SL2:mKate2) (SEQ ID NO: 57) using Gibson Assembly to generate ASJ332/pET1 (P_{srtx-1-b}:PINK-1:SL2:mKate2) (SEQ ID NO: 57). (FIG. 4)

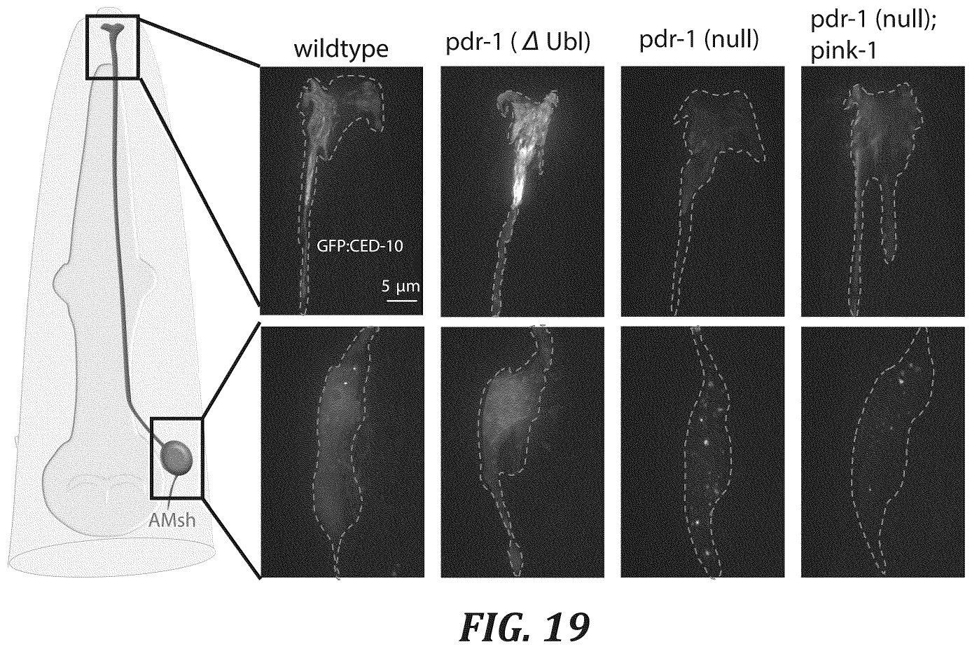

CED-10 plasmids: CED-10B isoform cDNA was isolated from a mixed stage cDNA library by PCR amplification with primers containing XmaI and NheI restriction enzyme sites and directionally ligated into pAS465 (P_{F53F4.13}: SL2: mCherry) to generate pAS275 plasmid (SEQ ID NO: 46). (FIGS. 11 and 19).

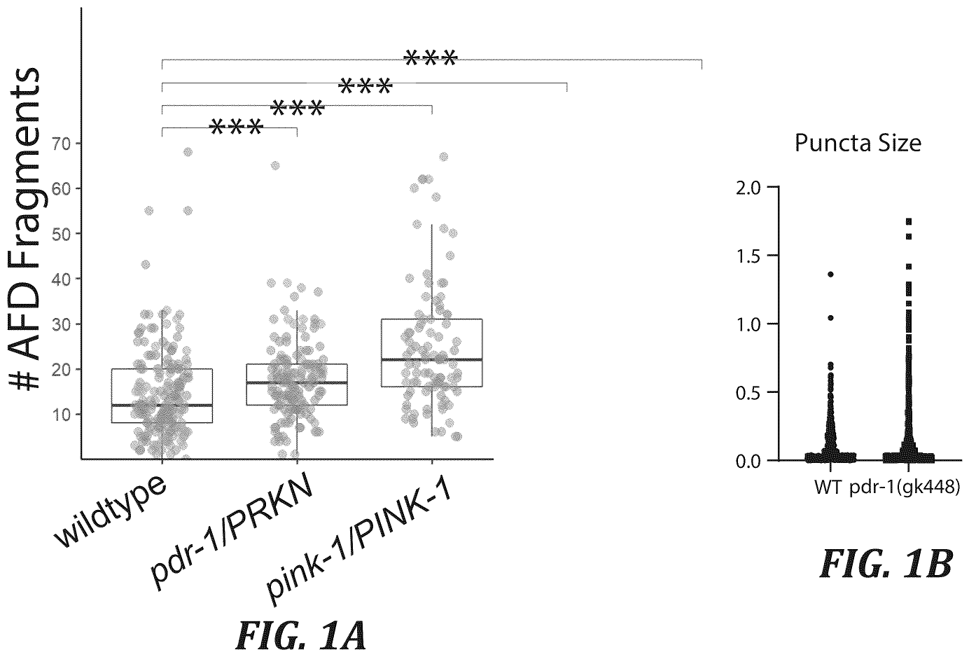

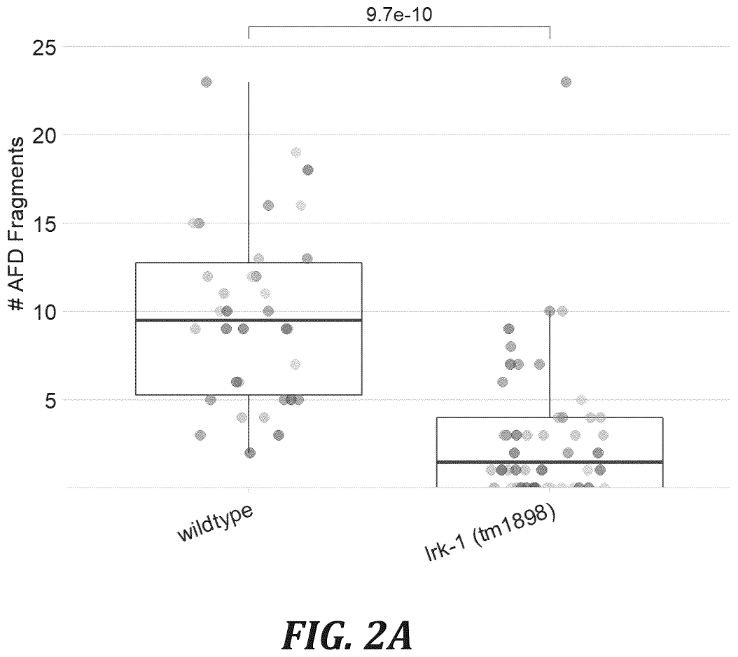

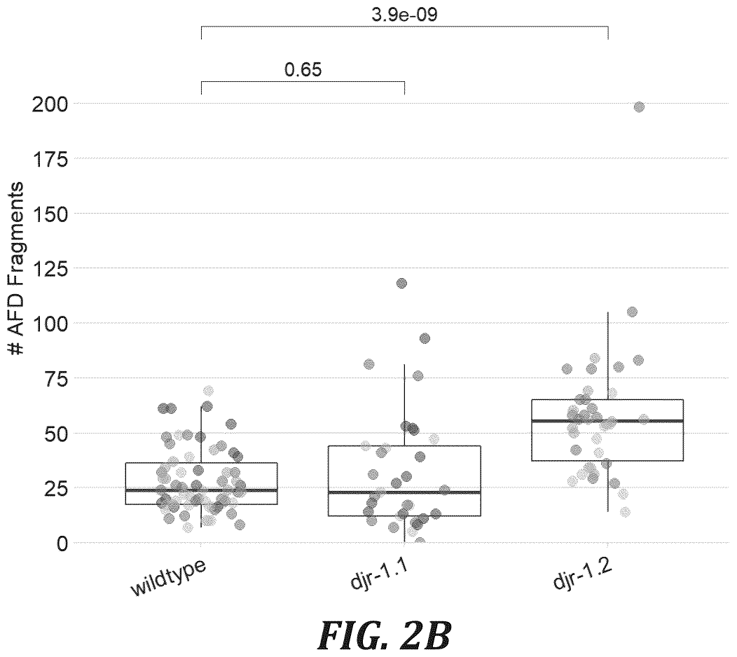

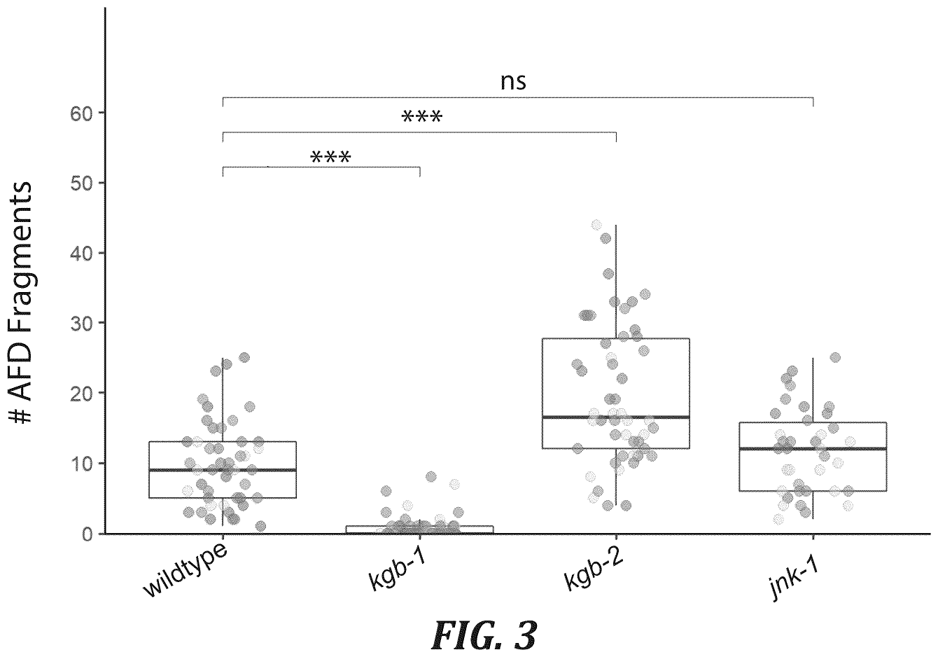

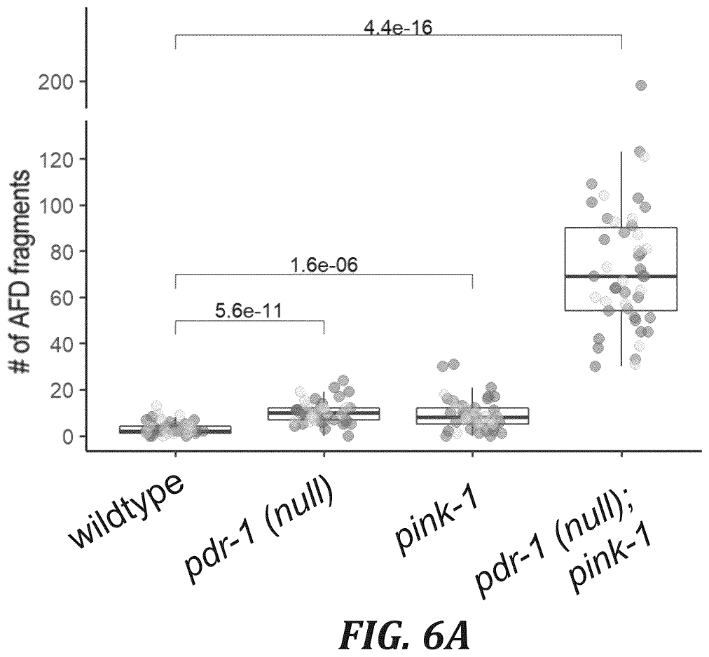

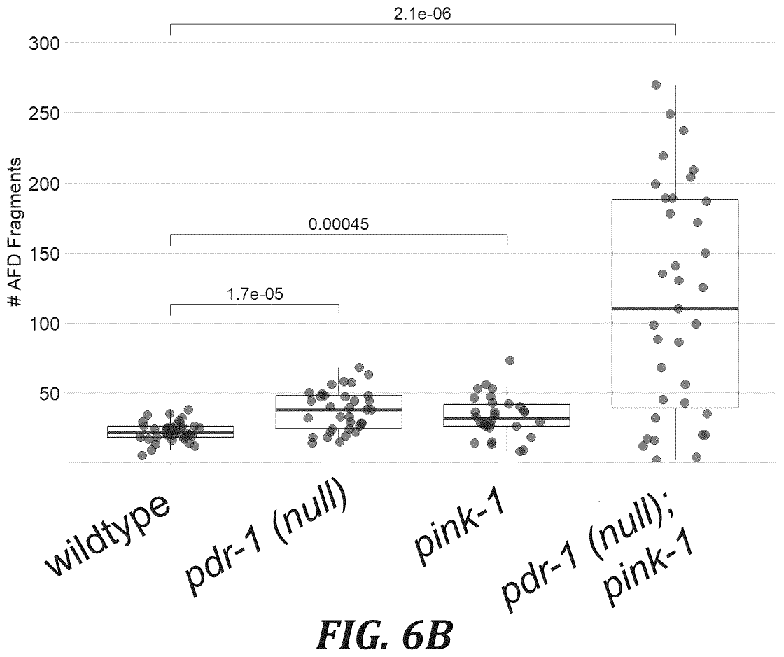

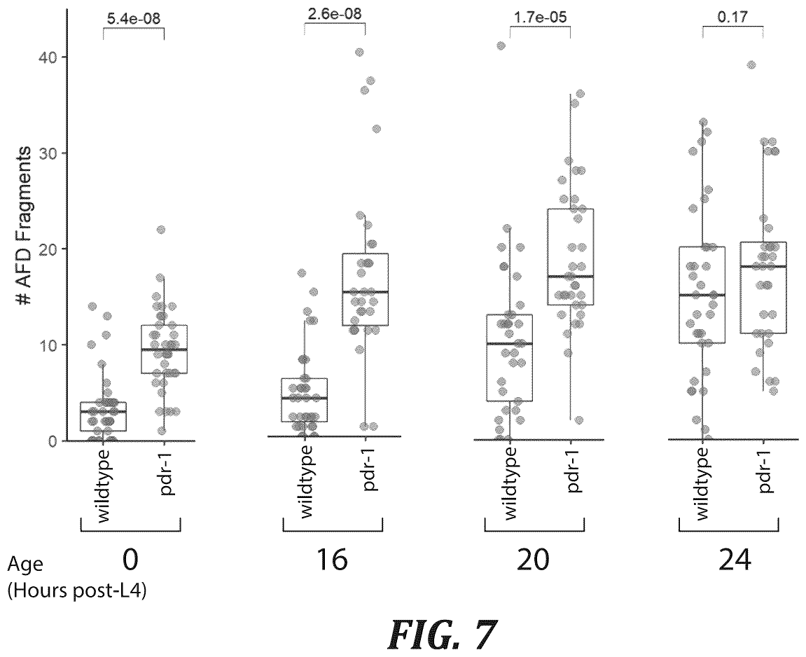

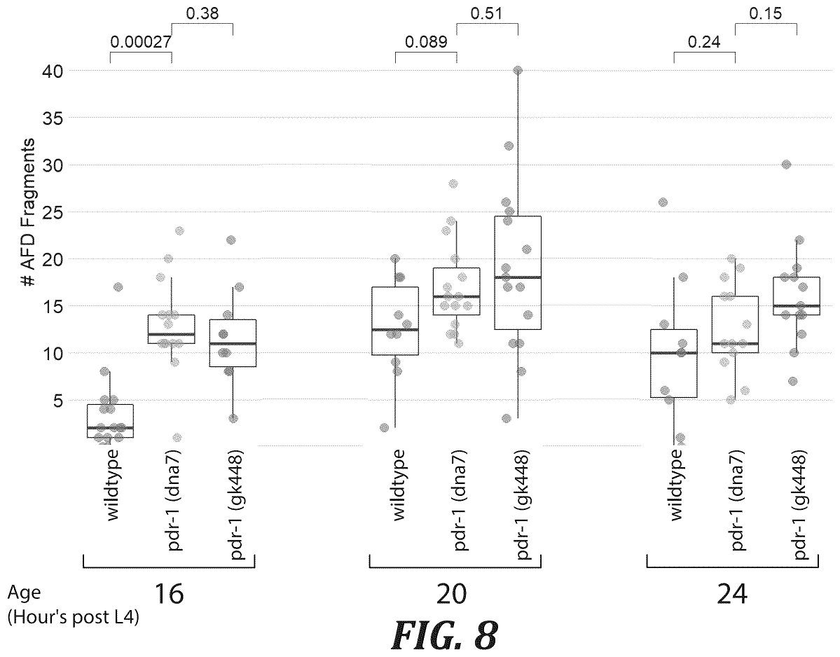

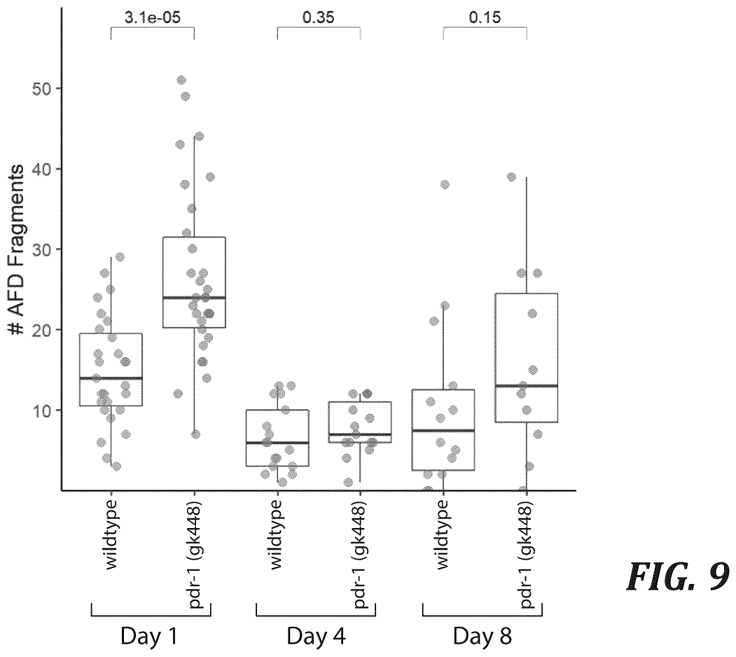

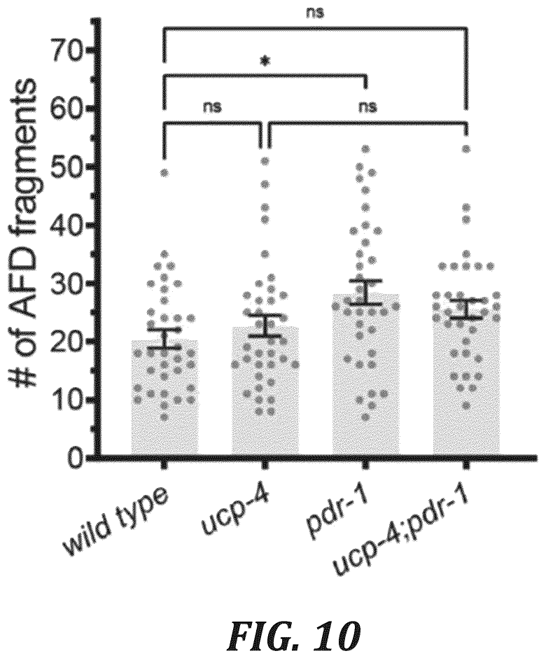

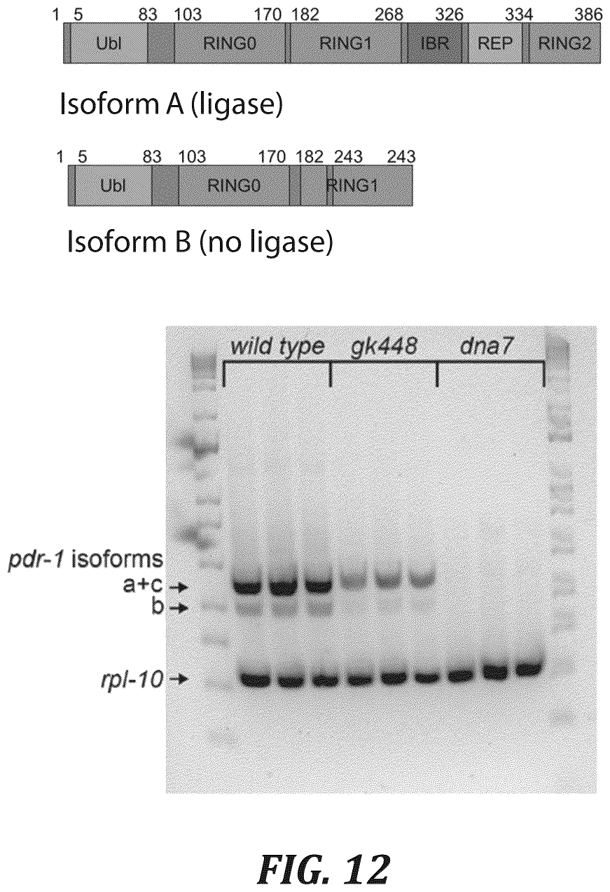

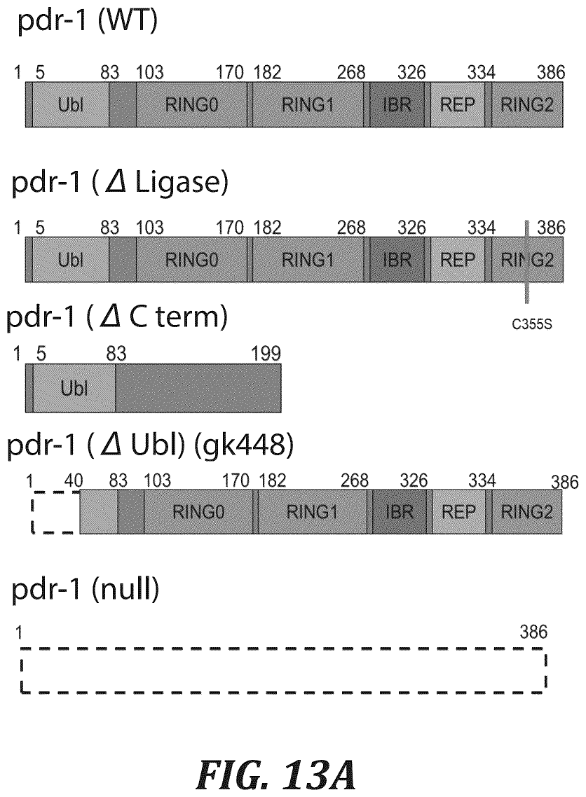

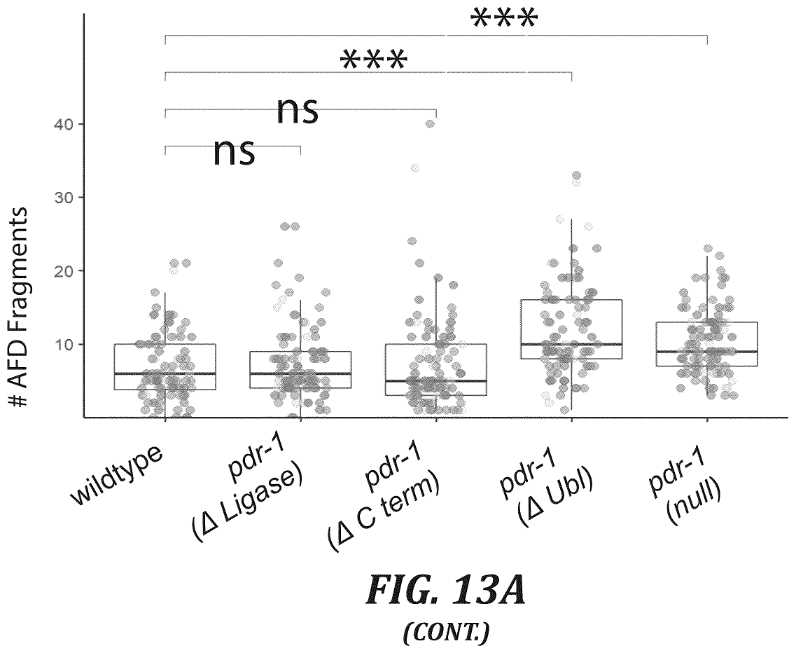

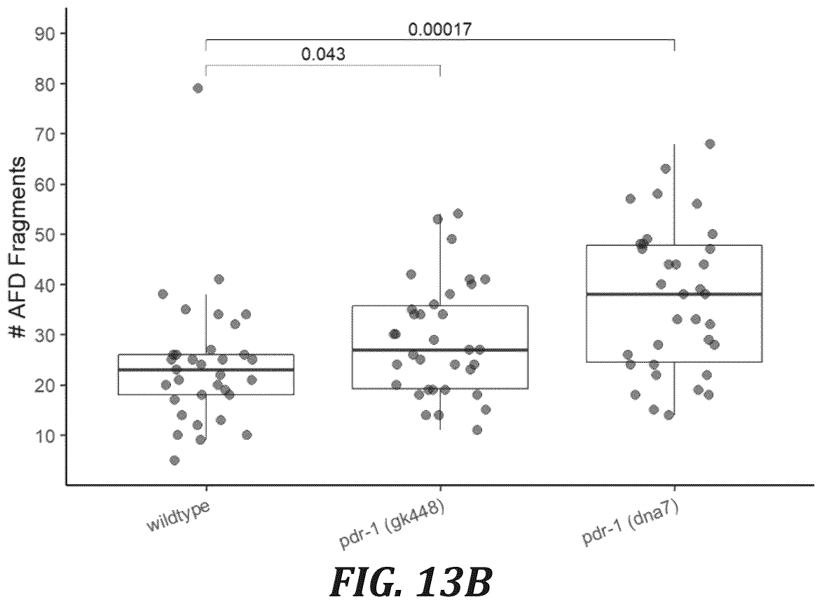

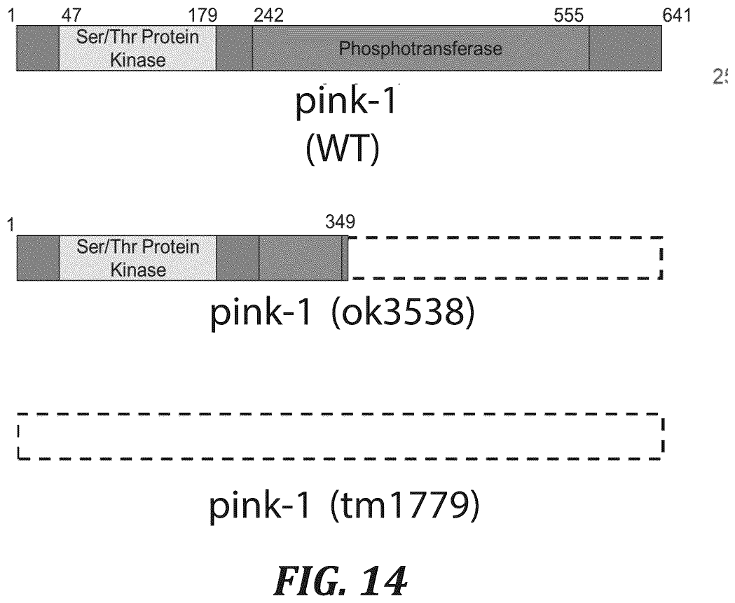

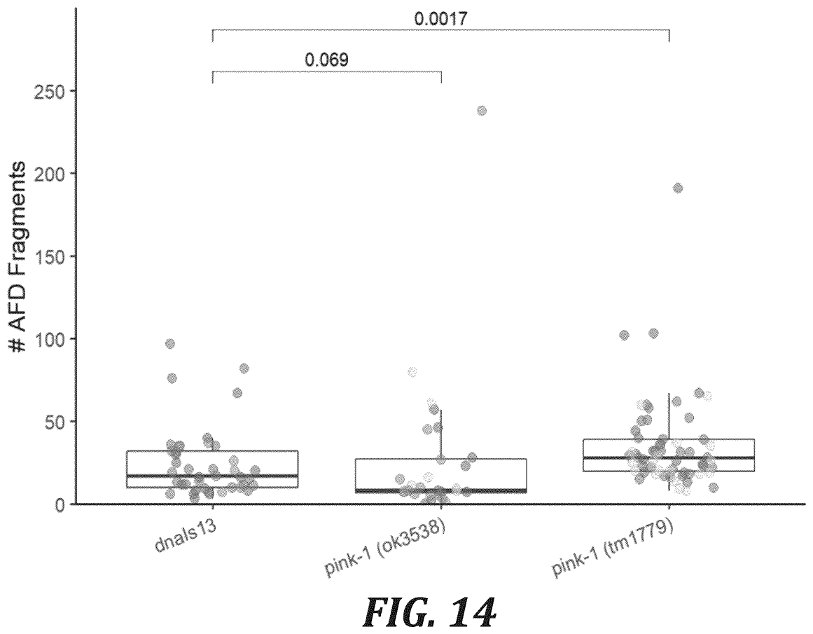

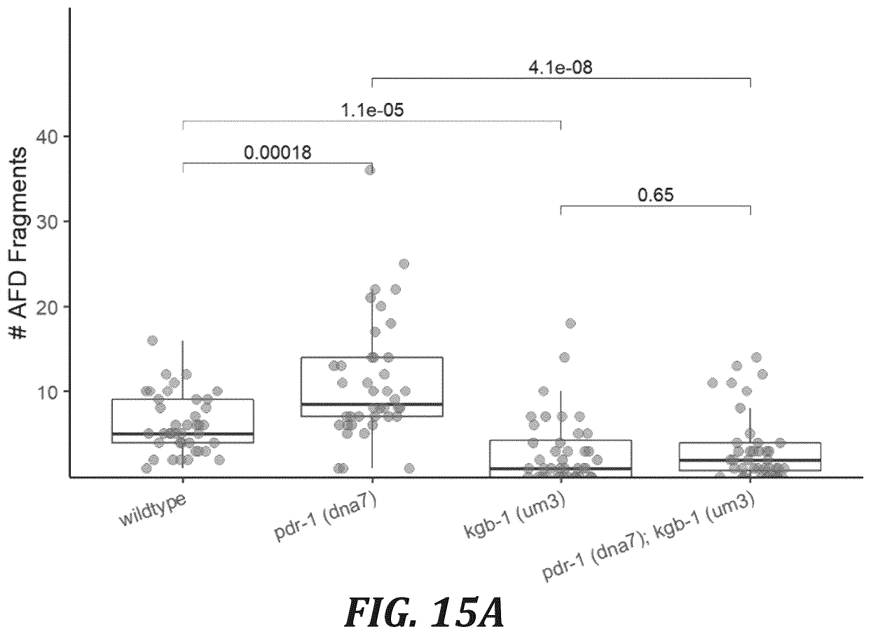

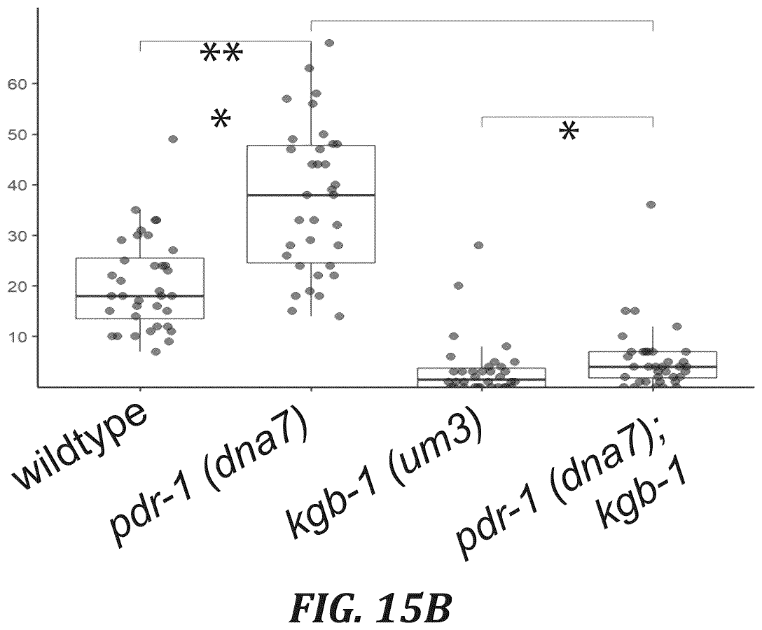

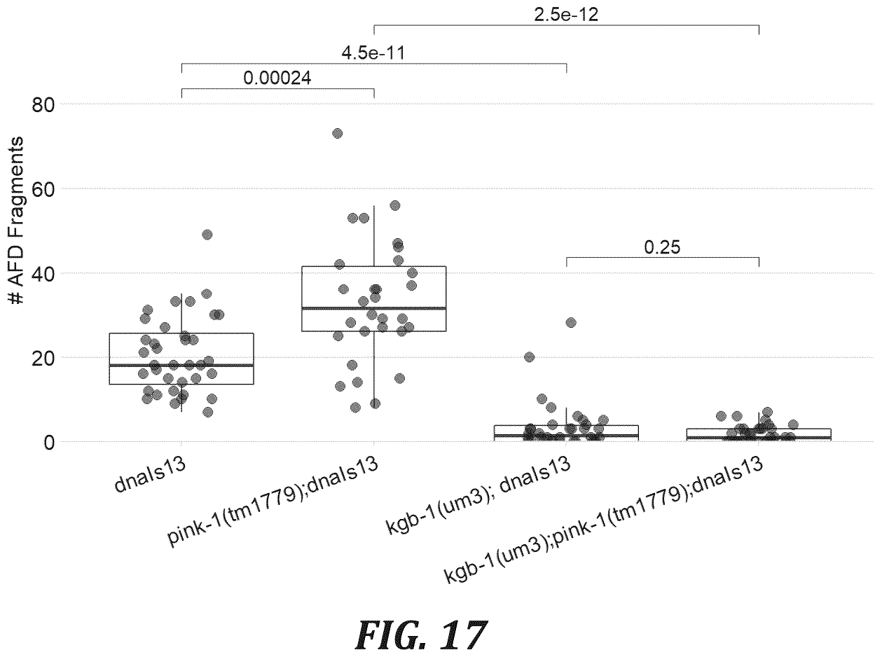

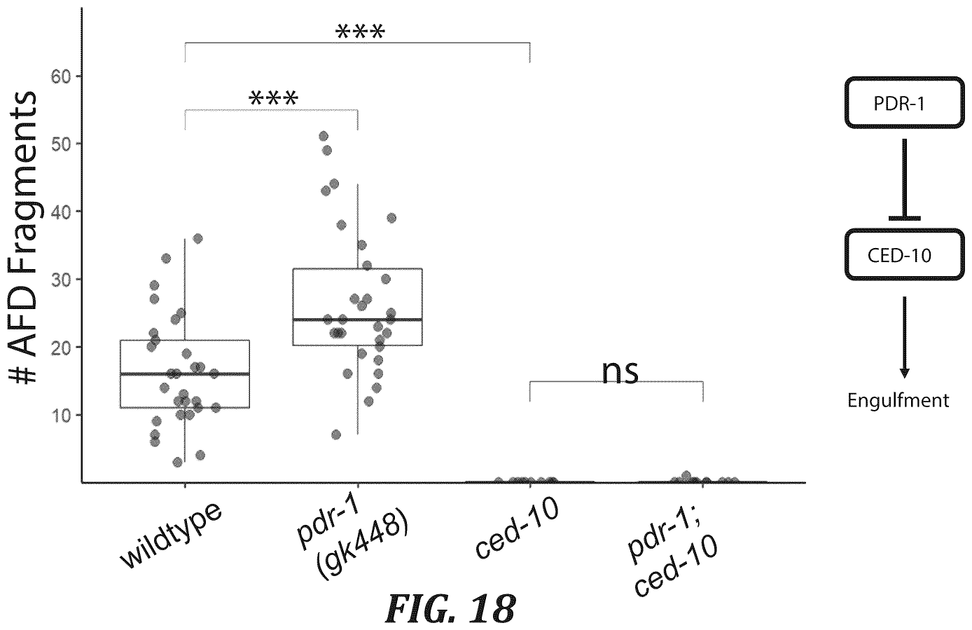

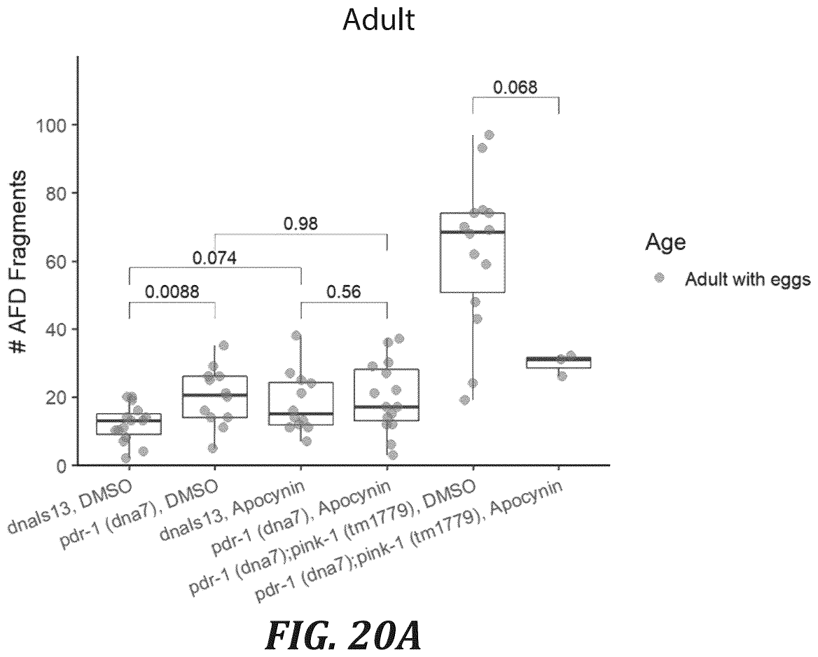

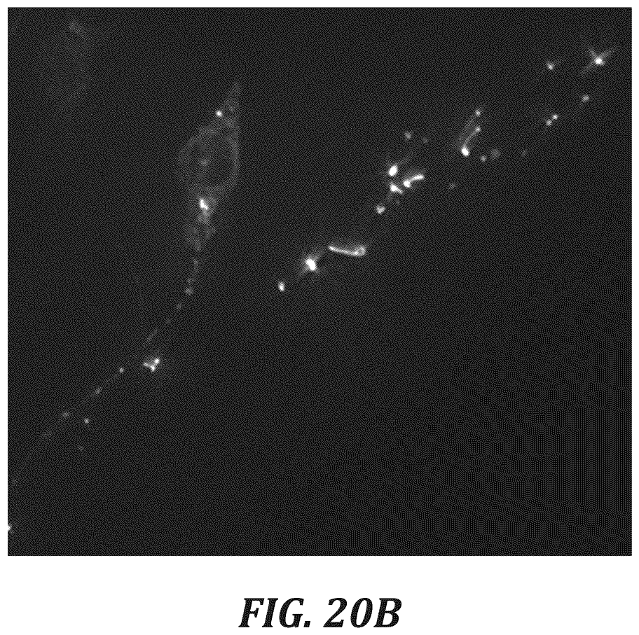

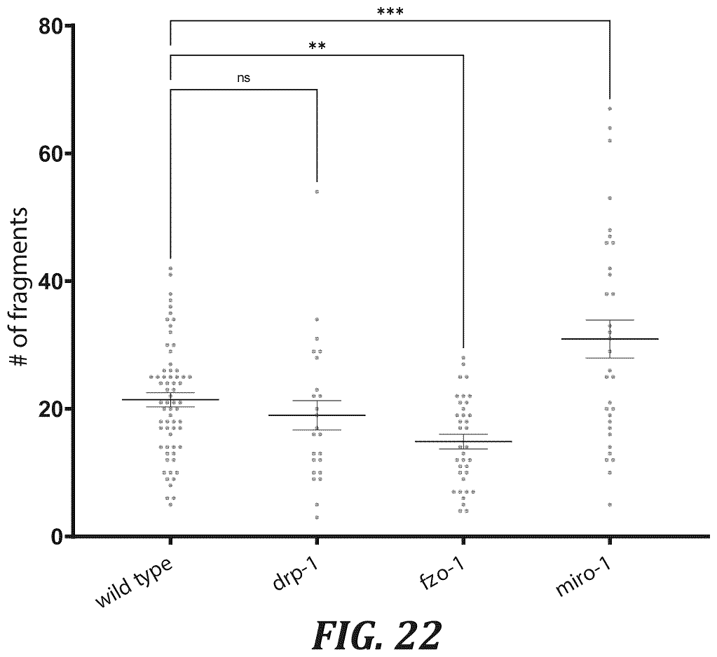

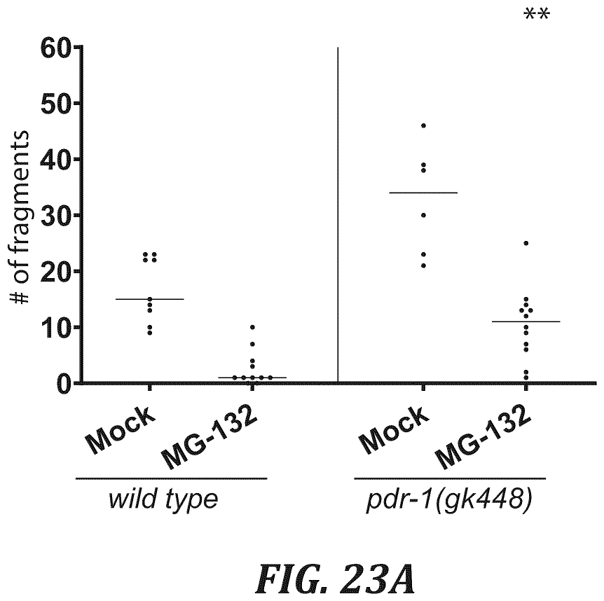

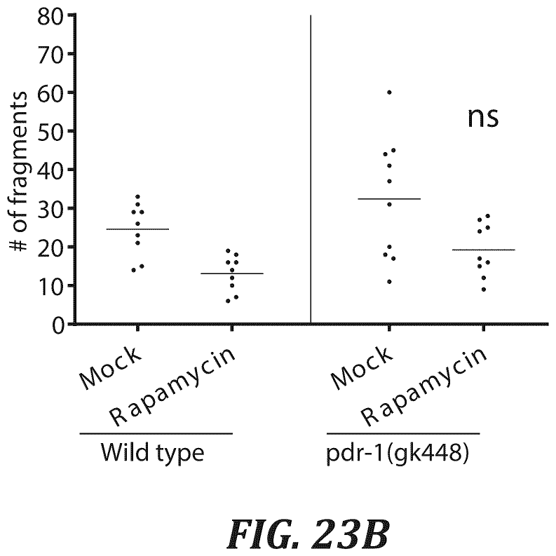

Recombineered fosmids: The following fosmids with GFP recombineered in-frame in the coding sequence were obtained from the MPI-TransgeneOme Project: gcy-8 (Clone ID: 02097061181003035 C08), and gcy-23 (Clone ID: 523378417130642 E08). (FIG. 1-23). Stable integrated transgenes of these were used to quantify number of engulfed fragments in glia.

UCP-4 constructs: UCP-4 cDNA from a mixed stage library was amplified by PCR and inserted in pAS465 to generate ASJ139/pSAR39.

PCR fragments containing 35 bp overlaps were generated from plasmids to generate amplicons with P_{F53F4.13}, GFP, CED-10, and UNC-54 3′ UTR. These amplicons were melted and re-annealed according to published protocols, and along with a crRNA specific to the MOSI transposon sequence injected into Day 1 adult worms. Transformants were screened via GFP fluorescence and confirmed with PCR and sequencing.

TABLE 2
Constructs

| | SEQ |
|---|---|
| Constructs | ID NO |
| pAS275 P53 CED-10B SL2 mCh | 46 |
| pAS516 pF53 GFP | 47 |
| ASJ 333 pET2 pF53F4.13 PINK-1 SL2 mKate2 | 48 |
| ASJ467 pET7 pF53-hPARK2-SL2-mCherry | 49 |
| pSAR1 F53F4.13 Sph-Not-Xma-ced-12 cDNA-Nhe-pSM- | 50 |
| SL2-cherry | |
| ASJ22 pSAR6 BamHI psr1 NheI SL2 mCh | 51 |
| ASJ29 pSAR8 pF53 XbaI psr1 AgeI mSc | 52 |
| ASJ72 pSAR19 pSAR20 SphI pSRTXB BamHI PDR-1 cDNA | 53 |
| SL2 mCh | |
| ASJ114 pSAR35 SphI pSRTX XmaI TAT-1a | 54 |
| ASJ139 pSAR39 pF53 XmaI ucp-4 cDNA NheI SL2 mCh | 55 |
| ASJ187 pSAR47 pF53 mKate2 PDR-1 | 56 |
| ASJ 332 pET1 Psrtx1-b pink1 SL2 mKate2 | 57 |
| pAS322 Psrtx-1b:pSM | |
| pAS464 Pf53F4.13:GFP | |
| pAS465 PF53F4.13:SL2:mCherry | |
| pAS548 PF53F4.13:SL2:mKate2 | |
| ASJ55 pSAR17 PF53F4.13:PDR-1:SL2: mCherry | |
| ASJ11 pSAR1 PF53F4.13:CED-12:SL2:mCherry | |

C. elegans Methods

C. elegans were cultured as known to one of skill in the art. Bristol N2 strain was used as wild type. For all experiments, animals were raised at 20° C. for at least two generations without starvation, picked as L4 larvae onto fresh plate, and assayed 1 day later unless otherwise noted. For analysis of glial engulfment, animals were crossed to the strain dnals13, an integrated transgene bearing gcy-23: GFP recombineered fosmid outcrossed to Bristol N2 four times. To control environmental variations, worms were raised from egg-lay in a humidity and temperature-controlled environment until imaging. Worms were staged at the L4 stage using vulval anatomy. Day 1 adult animals were staged at the L4 stage and then grown for approximately 24 more hours (unless otherwise stated) in the controlled environment. Worms were imaged and glial engulfment was analyzed as described in the methods below.

Germline transformations by microinjection to generate unstable extrachromosomal array transgenes were carried out using standard protocols known to those having ordinary skill in the art. Integration of extrachromosomal arrays was performed using UV+trimethyl psoralen.

Generation of CRISPR/Cas9 based mutations. For ligase dead pdr-1 allele: Using published protocols for the Alt-R CRISPR system, pdr-1 specific crRNAs, a repair template with 40 bp overhangs, rol-6 plasmid, and Cas9 mixture were injected into Day 1 adult worms. From the F1 generation, 12 rollers were picked to individual plates from each of the 2 highest transforming plates. 8 F2 animals from each of the 24 plates were lysed and the edit was confirmed with sequencing. For generation of pdr-1 deletion allele: Using a similar method to above, two crRNAs that bind to sequences flanking the pdr-1 genomic locus were used. Transformants were screened for successful deletion via PCR and confirmed via sequencing (FIGS. 5-10, 12, 13, 15, 16, 18, 19-23).

### Microscopy, Imaging, and Processing-Method 1

Animals were immobilized using either 2 mM tetramizole or 100 nm polystyrene beads. Images were collected on a Deltavision® Elite RoHS wide-field deconvolution system with Ultimate Focus® (GE), a PlanApo 60_/1.42 NA or OLY 100_/1.40 NA oil-immersion objective and a DV Elite CMOS Camera. Super-resolution microscopy images were collected on the Leica® VT-iSIM microscope or the Leica® SP8 confocal with Lightning. Images were processed on ImageJ, Adobe Photoshop® CC, or Adobe Illustrator® CC. Binning categories for population analyses were based on preliminary analyses of population distribution of puncta numbers/animal in wild type, and mutants with excess puncta (tax-2) or reduced puncta mutants (ced-10, psr-1). Preliminary analyses of these strains suggested that the bin intervals (0, 1-9, or 10+ puncta) are the most robust, conservative, and rapid assessment of phenotypes. Higher than 10 puncta/cell were not readily resolved without post-processing and therefore binned together in population scores. Some genotypes were selected for further post-hoc single-cell puncta quantification analyses. For this, glia puncta numbers were quantified using Analyze Particles function in ImageJ on deconvolved images. Individual puncta size measurements were done on yz orthogonal rendering of optical sections using 3D objects counter plug-in in ImageJ.

### Microscopy, Imaging, and Processing—Method 2

Worms were immobilized with 40 mM sodium azide. Images were collected on a Deltavision® Elite RoHS wide-field deconvolution system with Ultimate Focus® (GE), a PlanApo 60×/1.42 NA or OLY 100×/1.40 NA oil-immersion objective, and a DV Elite CMOS Camera. Images of the AFD puncta in the region of the AMsh were taken at 100× using FITC with 100% transmission and 0.0405 ms exposure with a reference image taken at 5% POL in the middle of the sample. For worms expressing an extrachromosomal array tagged with mCherry, images were also taken using an A594 filter with 0.02 ms exposure to ensure expression of the array in the cell of interest. Images were then deconvolved and cropped to 1024×1024 in the area of interest. Next, images were processed using FIJI®. All images were converted to 8-bit and brightness and contrast were set with a minimum of 0 and a maximum of 50. Next, a rectangle is drawn around the region of the AMsh adjacent to, but not including, the AFD cell body and excluding any area along the AFD process. Next, the image is cropped to this region, and the tool “3D Objects Counter” is run with the following settings: threshold=35; size min.=5 max.=7225848; exclude objects side. This number of puncta is then verified by eye and recorded.

### Electron Microscopy

Adult hermaphrodites were fixed in 0.8% glutaraldehyde-0.8% osmium tetroxide-0.1 M cacodylate buffer (pH 7.4) for 1 hr at 4° C. in the dark, and then quickly rinsed several times with 0.1 M cacodylate buffer. Animal heads were decapitated and fixed in 1% osmium tetroxide-0.1 M cacodylate buffer overnight at 4° C., quickly rinsed several times in 0.1 M cacodylate buffer, and dehydrated through a graded ethanol series. The samples were then embedded in Eponate 12 resin and polymerized overnight in a 60° C. oven. 70 nm ultrathin serial sections were collected onto pioloform-coated slot grids from the anterior tip of the animal to a distance of approximately 7 mm. Sections were examined on a JEOL 1400 TEM at an accelerating voltage of 120 kV. Images were acquired with a Gatan® Rio 4k_4k detector. Microvilli size measurements were done with ImageJ Measure Function on electron micrograph thin sections.

### Statistical Analyses—Method 1

Population puncta scoring was statistically analyzed using Fisher's exact statistical test in GraphPad Prism® 8. Puncta images were quantified using Analyze Particles function in Image J and analyzed with a one-Way ANOVA® with multiple-comparison test in GraphPad Prism® 8.

### Statistical Analyses—Method 2

Data and statistics were either graphed and analyzed using Prism9 or by using R. Statistical analysis in R was conducted using the open-source data visualization package ggplot2. Data is represented in a boxplot with each point representing the number of puncta engulfed within the cell body of one AMsh glia in a single worm. Dots represent the biological replicate in which the individual worm was imaged unless otherwise specified to represent genotype. Statistical analysis between groups shows a comparison of means using the nonparametric test of either the Wilcox rank sum test for two groups or a Kruskal Wallis test if there are more than two groups.

### Chemogenetic Silencing and RNAi

For chemogenetic silencing assays, 10 mM histamine (Sigma®) was added to NGM agar plates. L4 larval stage transgenic worms expressing HisCl1 in AFD were grown for 24 hrs. on either normal or histamine plates and assayed as day 1 adults. Plasmids expressing double-stranded RNA were obtained from the Ahringer Library. The L4440 empty vector was used as negative control. RNAi was performed by feeding synchronized L1 animals RNAi bacteria. L4 larva were moved to a fresh plate with RNAi bacteria and scored 24 hr later for glial puncta (nsIs483) or AFD-NRE defects (nsIs645).

RT-PCR, qPCR

Primers for RT-PCR were designed manually. Total RNA from bleach synchronized Day 1 adults was isolated, normalized at 1 μg between samples, and reverse transcribed into cDNA using ThermoFisher cDNA Reverse Transcription Kit. PCR was performed on cDNA libraries and analyzed by gel electrophoresis. Specific bands were isolated via cutting and DNA was isolated by gel extraction. The same forward primer used in the RT-PCR was used to sequence each band. qPCR primers were designed using the ThermoFisher TaqMan® Real-Time PCR tool. RNA was extracted and converted to cDNA as described above. qPCR was performed on an ABI® QuantStudio® 5 Real-Time PCR system and analyzed in the ABI® QuantStudio® 5 software.

### Example 2

C. elegans Glia Engulf Fragments of the AFD-NRE

Glia of the nematode C. elegans share molecular, morphological, and functional features with vertebrate sense-organ glia and astrocytes. AMsh glia-AFD neuron pair is a tractable experimental platform to define molecular mechanisms of single glia-neuron interactions. The AFD-NRE comprises ~45 actin-based microvilli and a single microtubule-based cilium that are embedded in the AMsh glial cell. An adherens junction between the AFD-NRE base and the AMsh glial cell isolates this glia-NRE compartment.

Upon imaging fluorescently labeled AFD-NREs in transgenic animal strains, labeled fragments were consistently observed to be disconnected from the neuron. Reconstructions-based FIB-SEM serial section data also revealed AFD-NRE fragments disconnected from the rest of the AFD neuron. Further examination using two-color imaging revealed that many of these fragments reside within the AMsh glial process and cell body. To confirm that these glial puncta do not reflect spurious reporter protein misexpression in glia but rather derive from the AFD, AFD neurons were ablated early in larval development and puncta on the first day of adulthood was sought. Upon ablation of one of the two bilateral AFD neurons by laser microsurgery in first larval stage (L1) animals, fragment formation was blocked on the operated side, but not on the unoperated side, or in mock-ablated animals. Similar results were seen with stochastic genetic ablation of AFD using the pro-apoptotic BH3-domain protein EGL-1, expressed using an embryonic AFD-specific promoter. Therefore, AMsh glia engulf fragments of the AFD-NRE in C. elegans.

3D super-resolution microscopy studies revealed that the average size of AFD-derived glial puncta is 541±145 nm along their long (yz) axis. These fragments are an order of magnitude smaller than other exophers extruded from neurons exposed to cellular stress (~3.8 mm in diameter) and larger than ciliary extracellular vesicles (~150 nm). This size is of the same order of magnitude as the sizes of individual AFD-NRE microvilli or cilia as measured by electron microscopy.

### Example 3

### AMsh Glia Engulfment of AFD-NREs Occurs in Adults

Engulfment of neuronal fragments by glia has been suggested to refine neuronal circuit connectivity during neural development. Post development, glial engulfment is thought to regulate animal behaviors and memory. To determine when C. elegans AMsh glia initiate engulfment of AFD-NRE fragments, engulfed NRE puncta at different life stages were counted. These puncta are rarely found in embryos or early larval stages, but are easily detected in L4 larvae and increase in numbers during adulthood. Thus, consistent with L1 laser ablation studies, engulfment of AFD-NREs by glia occurs after development of the AFD-NRE is largely complete.

Approximately 65% of 1-day old adult animals expressing the AFD-NRE-specific markers including gcy-8: GFP or gcy-23: GFP raised at 20° C. have AMsh glia containing >10 puncta, and another ~32% of animals have 1-9 puncta/glia (n=171). The AMsh glial cell of 1-day-old adults has on average 14±1 puncta (n=78). Using time-lapse microscopy, individual puncta separate from the NRE at a frequency of 0.8±0.3 events/min and travel at 1.05±0.1 mm/s down the glial process towards the cell body, consistent with motor-protein-dependent retrograde trafficking (quantifications of videos from n=5 animals). Finally, age-matched animals raised at different cultivation temperatures differ in glia puncta accumulation.

### Example 4

### Nuclear Localization Regulates Pruning

A nuclear role for PDR-1 in glia or in regulation of glial pruning has not been previously described. Pruning is inhibited when PDR-1 becomes localized into the glial nucleus. Restricting PDR-1 out of the nucleus does not alter pruning rates. Increased levels of CED-10/Rac1 translocate PDR-1/Parkin to glial nuclei. Altered levels of CED-10/Rac1 regulate rates of pruning. Thus, dependent regulation of PDR-1 and CED-10 regulates PDR-1 localization to the nucleus and glial pruning. (FIG. 11B).

### Example 5

### Effects of Exposure to Drugs on Glial Pruning

4 mL standard C. elegans nematode growth medium (NGM) was aliquoted into 35 mm Petri dishes and stored at 4 degrees Celsius. Plates were warmed to room temperature and seeded with 100 μL of OP50 and dried overnight. Plates were then spotted with 100 μL of diluted SP600125 (in DMSO), Apocynin (in DMSO), or EHop-016 (in DMSO) for a final concentration of 0.5% or 1% DMSO (as labeled) and a final concentration of the treatment drug of 50 μM (unless otherwise specified). Spot treatment was allowed to dry before L1 staged C. elegans were transferred onto the plate. Worms were then imaged approximately 2 days later. Worms were staged at this time by vulval anatomy, body size, and the presence or absence of eggs. These stages were noted for the worms and then data was subset to compare the effects of drugs at different stages. At this time, worms were imaged, and glial engulfment levels were analyzed.

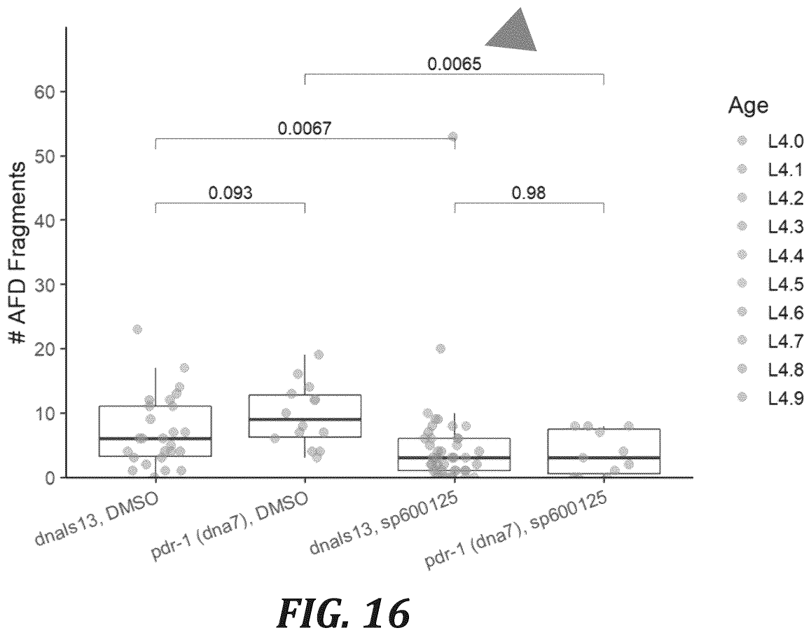

Results of these drug treatments are shown in FIGS. 16, 20, 21, and 23. Briefly, genetic loss of CED-10/Rac1 or JNK, or their activity modulation by drug treatments, can modify glial engulfment of wild type, as well as pdr-1 and pink-1 mutations. Thus, modifying Rac1 or JNK activity is relevant to mitigating glial engulfment defects seen in animal mutants for PD-associated genes.

### CED-10/Rac1

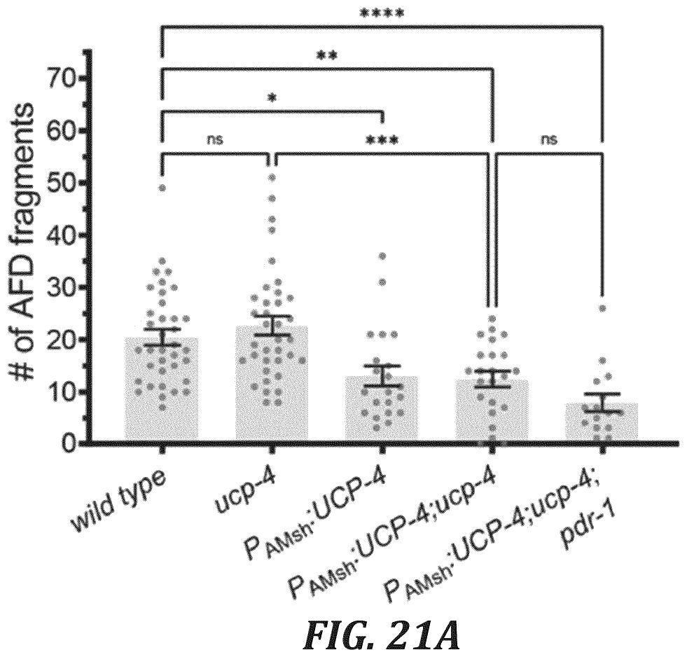

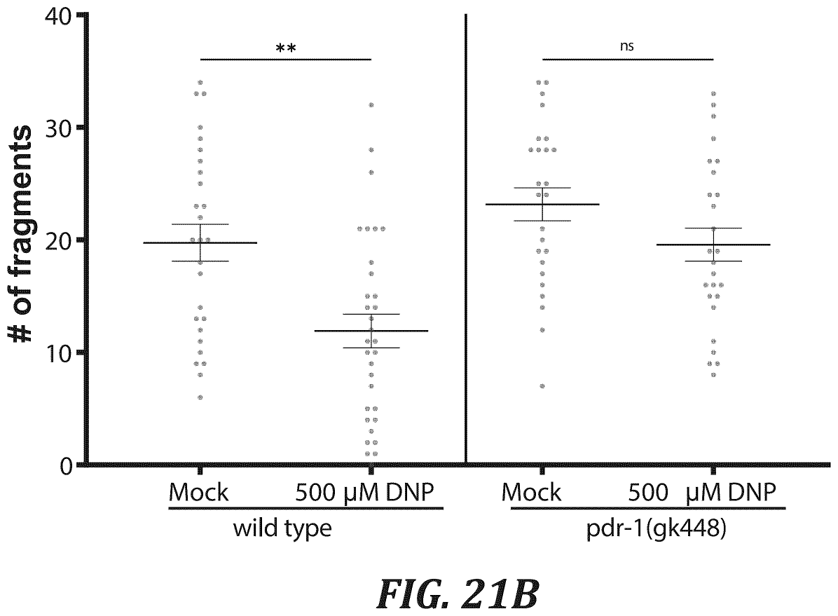

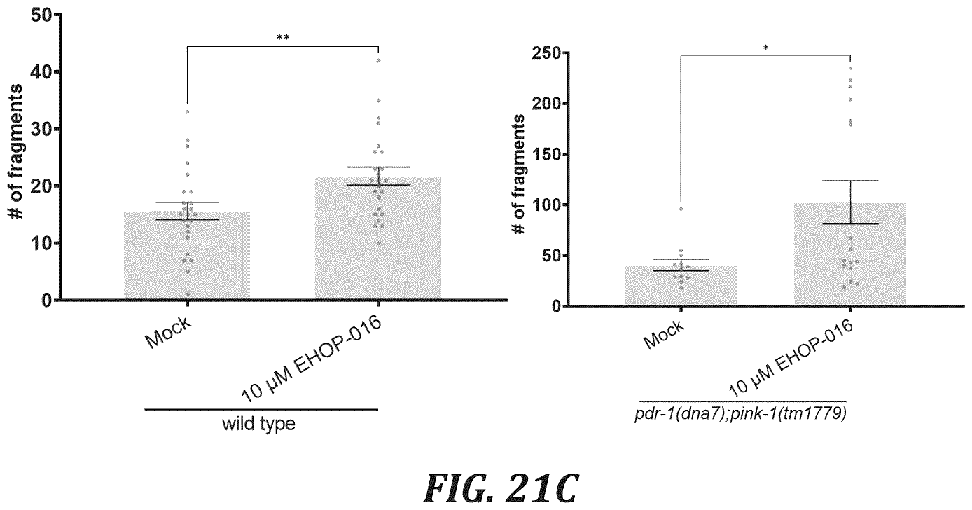

Drugs which inhibit CED-10/Rac1 function, without losing gene product, increase pruning similar to loss of the pdr-1 gene. For example, exposure to 10 μM of the drug EHOP-016 increased pruning in wild type and pdr-1; pink-1 double mutant animals (FIG. 21C). Unlike mutation in ced-10/Rac1 or its inhibitor Apocynin, it did not suppress defects seen in pdr-1 single or pdr-1; pink-1 double mutant animals, as judged by the number of engulfed fragments in glia in each case, as seen in FIG. 21C. The variability in response properties of different methods of perturbing Rac1 activity suggests that specific regulation kinetics are required. Thus, manipulation of CED-10/Rac1 can alter pruning rates, and specific perturbations can exaggerate or suppress defects in glial pruning from loss of PDR-1.

While illustrative embodiments have been illustrated and described, it will be appreciated that various changes can be made therein without departing from the spirit and scope of the invention.

1. A method for regulating an aberrant glia-directed engulfment and active pruning of Neuronal Receptive Endings (NRE), the method comprising:
contacting a neuronal and/or a glia cell with an effective amount of a composition effective in modulating at least one gene and/or protein associated with glia-directed engulfment and active pruning of Neuronal Receptive Endings (NRE).

2. The method of claim 1, wherein the at least one gene and/or protein associated with glia-directed engulfment and active pruning of Neuronal Receptive Endings (NRE) comprises a gene in, and/or a protein expressed by, a neuronal cell, a glia cell, or a combination thereof.

3. The method of claim 1, wherein the at least one gene and/or protein associated with glia-directed engulfment and active pruning of Neuronal Receptive Endings (NRE) is selected from: P4-type ATPase/ATP8A, phosphatidyl serine receptor (PSR-1), PAT-2/α-integrin, CED2/CrkII, CED5/DOCK1, CED-12/ELMO, PDR-1/PARKIN (E3 ubiquitin ligase), PTEN-induced putative kinase 1 (PINK1) PINK1/PARK6 (mitochondrial serine threonine kinase), UCP-4/SLC25A27, DJR-1.2/DJR, LRK-1/LRRK2, KGB-1/JNK, KGB-2/JNK and combinations thereof.

4. (canceled)

5. The method of claim 1, wherein the composition comprises a CED-10/Rac1 regulator or effector, a PDR-1/PARKIN regulator or effector, a PINK1/PINK regulator or effector, a LRK-1/LRRK regulator or effector, a DJR-1.2/DJR regulator or effector, a KGB-1/JNK regulator or effector, a KGB-2/JNK regulator or effector, a UCP-4/SLC25A27 regulator or effector, a CED-2/CrkII regulator or effector, a CED-5/DOCK1 regulator or effector, a CED-12/ELMO regulator or effector, and combinations thereof.

6. (canceled)

7. (canceled)

8. The method of claim 5, wherein the composition comprises EHOP-016, Sp600125, MG-132, rapamycin, apocynin, a pharmaceutically acceptable salt thereof, or a combination thereof.

9. (canceled)

10. The method of claim 1, wherein the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof, in a neuronal cell, and wherein the protein comprises P4-type ATPase/ATP8A or an isoform thereof.

11. (canceled)

12. The method of claim 1, wherein the composition is effective in modulating the gene encoding the at least one protein, an expression of the at least one protein, an activity of the at least one protein, localization of the at least one protein, a regulator, a downstream effector of the at least one gene and/or the at least one protein, or a combination thereof, in a glia cell, and wherein the at least one gene and/or protein is selected from PSR-1, PAT-2/α-integrin, PINK-1/PINK, PDR-1/PARKIN, LRK-1/LRRK2, DJR-1.2/DJR, UCP-4/SLC25A27, CED-10/RAC1, KGB-1/JNK, KGB-2/JNK, a mutant thereof, an isoform thereof, and combinations thereof.

13. (canceled)

14. (canceled)

15. The method of claim 12, wherein the at least one gene and/or protein is PDR-1/PARKIN, a truncated PDR-1/PARKIN, or isoforms thereof,
wherein the truncated PDR-1/PARKIN comprises a PDR-1/PARKIN protein lacking a Ubl domain, and
wherein the composition is effective in restoring expression of a PDR-1/PARKIN comprising the Ubl domain in the glial cell.

16. (canceled)

17. (canceled)

18. The method of claim 15, wherein the composition comprises a modulator of the activity of Janus N-terminal Kinase (JNK) or an isoform thereof, and wherein the JNK or the isoform thereof is a downstream effector of PDR-1/PARKIN.

19. The method of claim 15, wherein the composition is effective in modulating the expression levels, localization, or the activity of Rac1 GTPase (CED-10/RAC1), and wherein the Rac1 GTPase (CED-10/RAC1) is acting with PDR-1/PARKIN.

20. The method of claim 15, wherein the composition is effective in modulating the expression of the mitochondrial uncoupling protein UCP-4 in the glia cell, wherein the composition comprises an expression vector encoding UCP-4 or modulator thereof, and wherein the composition is effective in modulating PDR-1/PARKIN.

21. The method of claim 15, wherein the method is effective in modulating the timing of the glia-mediated NRE engulfment, and wherein the method is effective in preventing pre-mature onset of glia-mediated NRE engulfment.

22. The method of claim 12, wherein the at least one gene and/or protein is PINK-1, a mutated PINK-1 protein, or a combination thereof, and
wherein the composition is effective in restoring a serine/threonine kinase function of PINK-1 protein.

23. (canceled)

24. The method of claim 22, wherein the composition comprises a regulator of the activity of Janus N-terminal Kinase (JNK), or an isoform or ortholog thereof, and wherein the Janus N-terminal Kinase (JNK), or the isoform or ortholog thereof, is a modulator of PINK-1.

25. The method of claim 22, wherein the composition is effective in regulating expression levels, localization, or activity of Rac1 GTPase (CED-10/RAC1), and wherein Rac1 GTPase (CED-10/RAC1) is acting with PINK-1.

26. (canceled)

27. (canceled)

28. The method of claim 1, wherein the method is effective in regulating neuronal function, neuronal shape, an alteration in the rate of glia-directed engulfment and active pruning of Neuronal Receptive Endings (NRE), timing of glia-directed engulfment and active pruning of Neuronal Receptive Endings (NRE), or a combination thereof.

29. The method of claim 1, wherein the method is effective in treating a neurodegenerative disorder or disease in a subject in need thereof, and
wherein the neurodegenerative disorder or disease is selected from Parkinson's disease (PD), Alzheimer's disease, Autism Spectrum Disorders, leprosy, and gliomas.

30. (canceled)

31. (canceled)

32. The method of claim 1, wherein the composition further comprises a pharmaceutically acceptable carrier or excipient.

33. A method of treating a neurodegenerative disease in a subject in need thereof, the method comprising administering to the subject an effective amount of a therapeutic composition effective in modulating at least one gene and/or protein associated with glia-directed active pruning of Neuronal Receptive Endings (NRE).

34.-58. (canceled)

59. A method for treating Parkinson's Disease in a subject in need thereof, the method comprising administering to the subject a therapeutically effective amount of a composition effective in modulating at least one gene and/or protein associated with Parkinson's Disease (PD), wherein the gene and/or protein is expressed in a glia cell.

60.-87. (canceled)
